(19) United States
(12) Patent Application Publication
(10) Pub. No.: US 2024/0350261 A1
(43) Pub. Date: Oct. 24, 2024

(54) PROSTHETIC HEART VALVE

(71) Applicant: EDWARDS LIFESCIENCES CORPORATION, Irvine, CA (US)

(72) Inventors: Alon Ben-Yosef, Ramot Menashe (IL); Eran Grosu, Kfar-Saba (IL); Michael Bukin, Pardes Hanna (IL); Nikolai Gurovich, Hadera (IL); Anatoly Dvorsky, Haifa (IL); Noam Miller, Givatayim (IL); Ilan Leshecz, Hadera (IL); Joseph Mordechai Leichner, Netanya (IL)

(21) Appl. No.: 18/761,651

(22) Filed: Jul. 2, 2024

Related U.S. Application Data
(63) Continuation of application No. PCT/US2023/010425, filed on Jan. 9, 2023.
(60) Provisional application No. 63/404,675, filed on Sep. 8, 2022.
(60) Provisional application No. 63/298,526, filed on Jan. 11, 2022.

(51) Int. Cl. A61F 2/24

(52) U.S. Cl. CPC A61F 2/2418; A61F 2220/0033; A61F 2230/0006; A61F 2250/001

(57) ABSTRACT

This disclosure is directed to prosthetic heart valves having frames with features configured to increase the flexibility of certain frame components and/or to allow the frame to maintain an at least substantially cylindrical shape during deployment. In one example, a frame for an implantable device can include a first end, a second end, a plurality of angled struts, and a plurality of vertical struts. One or more vertical struts of the plurality of vertical struts includes a fixed end portion connected to a vertical strut junction, a free end portion, and a plurality of apertures disposed between the fixed end portion and the free end portion and configured to increase the flexibility of the one or more vertical struts. In some examples, one or more vertical struts of the plurality of vertical struts comprises an axially compressible aperture configured to enable the axial compression of the strut.

### CROSS-REFERENCE TO RELATED APPLICATIONS

This application is a continuation of International Application No. PCT/US2023/10425, filed Jan. 9, 2023, which claims the benefit of U.S. Provisional Patent Application No. 63/404,675, filed on Sep. 8, 2022, and U.S. Provisional Patent Application No. 63/298,526 filed on Jan. 11, 2022, all of which are incorporated by reference herein in their entireties.

### FIELD

The present disclosure relates to implantable expandable prosthetic heart valves and frame structures for use with expandable prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a delivery capsule of the delivery apparatus so that the prosthetic heart valve can self-expand to its functional size.

### SUMMARY

When prosthetic heart valve frames are retained in a radially compressed configuration, the varying strength and stiffness of the various portions of the frame may cause some portions of the frame to radially compress less than other portions of the frame in a phenomenon sometimes called “barreling”. Disclosed herein are prosthetic heart valve frames having features for ameliorating the tendency of the frames to experience “barreling” under compression, or to cause bending of actuators used with the frames. In some examples, the features are apertures designed to increase the flexibility of certain frame components to reduce the bending forces that the components may apply to actuators when the frame is in a radially compressed configuration. In some examples, various components of the frame may be plastically deformed or heat set into a position that reduces the magnitude of bending imparted to any connected actuators. In some examples, the frame comprises additional struts configured to translate compressive forces more evenly across various frame components reduce the severity of “barreling”. Also disclosed herein are methods for deploying prosthetic heart valves containing frames incorporating any or all of these features.

A prosthetic heart valve includes a frame (which can also be referred to as “a stent” or “a support structure”) and a valve structure (for example, leaflets) configured for regulating the flow of blood in one direction. In addition to these components, a prosthetic heart valve can comprise one or more of the components disclosed herein.

In some examples, a prosthetic heart valve can comprise an axially extending first post with one or more apertures extending therethrough, wherein the one or more apertures increase the radial flexibility of the axially extending first post.

In some examples, a prosthetic heart valve can comprise a window configured to receive a component of an actuator in the axially extending first post.

In some examples, a prosthetic heart valve can comprise one or more apertures positioned between a first end portion of the axially extending first post and the window.

In some examples, a prosthetic heart valve can comprise one or more apertures positioned between a second end portion of the axially extending first post and the window.

In some examples, and a prosthetic heart valve can comprise an axially extending first post, connected at a first end portion to a junction formed by two or more struts of the frame, and connected at the second end portion to one or more struts of the frame by a laterally-extending member extending between the second end portion and another strut of the frame.

In some examples, a prosthetic heart valve can comprise one or more heat-set components configured to relieve the bending strains on one or more actuators.

In some examples, a prosthetic heart valve can comprise an axially extending first post with one or more reinforcing struts extending from an end portion of the first post to one or more other components of the frame.

In some examples, a prosthetic heart valve can comprise an axially extending first post with an axially collapsible aperture.

In some examples, the axially extending first post with an axially collapsible aperture can be a mechanical fuse.

In some examples, a prosthetic heart valve comprises one or more of the components recited in Examples 1-208 disclosed herein.

Certain examples concern a frame for an implantable device, comprising a first end, a second end, a longitudinal axis extending from the first end to the second end, a plurality of angled struts non-parallel to the longitudinal axis, and a plurality of vertically oriented struts extending parallel to the longitudinal axis and coupled to the angled struts at one or more vertical strut junctions. One or more vertical struts of the plurality of vertically oriented struts includes a fixed end portion connected to a vertical strut junction, a free end portion, and a plurality of apertures disposed between the fixed end portion and the free end portion and configured to increase the flexibility of the one or more vertical struts.

Certain examples concern a prosthetic heart valve, comprising a frame having a first end, a second end, a longitudinal axis extending from the first end to the second end, a plurality of angled struts extending transverse to the longitudinal axis, a plurality of vertical struts extending parallel to the longitudinal axis, and a plurality of junctions formed by an intersection of two or more angled struts, vertical struts, or a combination thereof. The prosthetic heart valve also includes a valvular structure having a plurality of leaflets configured to allow blood to flow through the prosthetic heart valve from the first end of the frame to the second end of the frame and to prevent blood from flowing through the prosthetic heart valve from the second end of the frame to the first end of the frame. At least one vertical strut of the plurality of vertical struts has a fixed end and a free end, is attached at the fixed end to one junction of the plurality of junctions and extends radially inwards from the diameter of the frame with a first radial displacement when the frame is in a radially expanded configuration.

Certain examples concern a medical assembly, comprising a radially expandable annular frame having a distal end, a proximal end, a vertical axis extending from the distal end to the proximal end, a plurality of interconnected non-actuated struts, and at least one actuated strut, an actuator configured to radially expand the frame from a radially compressed configuration to a radially expanded configuration. There is a commissure opening in at least one of the plurality of non-actuated struts, and at least one actuated strut comprises a fixed end connected to one or more of the plurality of non-actuated struts, a free end, and a channel extending from the fixed end to the free end configured to receive a first component of the actuator. The non-actuated strut having the commissure opening extends radially inwards with a first radial displacement from the adjacent non-actuated struts while the annular frame is in the radially expanded configuration.

Certain examples concern an implantable stent, comprising a distal end, a proximal end, a longitudinal axis extending between the distal end and the proximal end, a plurality of angled struts defining an annular body, a plurality of vertical struts extending parallel to the longitudinal axis, and a plurality of junctions formed by an intersection of two or more angled struts or vertical struts. At least one vertical strut of the plurality of vertical struts comprises a first end attached to a first junction of the plurality of junctions, a body extending from the junction parallel to the longitudinal axis, and a second end at the opposite end of the body from the first end.

Certain examples concern a medical assembly, comprising a frame having a first end portion, a second end portion, a longitudinal axis extending between the first end and the second end, a plurality of interconnected angled struts extending transverse to the longitudinal axis, a plurality of vertical struts extending parallel to the longitudinal axis, an actuator, and a commissure opening. The actuator is configured to radially expand the frame from a radially compressed configuration to a radially expanded configuration. The plurality of interconnected angled struts and the plurality of vertical struts define a plurality of inner frame cells and a plurality of outer frame cells each having a distal apex and a proximal apex. At least one vertical strut of the plurality of vertical struts comprises a first strut end, a second strut end, and at least one aperture. The vertical strut is configured to receive a first component of the actuator, is connected at the first strut end to the distal apex of an inner frame cell of the plurality of inner frame cells, extends toward the proximal apex of the inner frame cell, is connected at the second end to a portion of the inner frame cell, and is heat set to deflect radially inwards from the inner frame cell while the frame is in the radially expanded configuration. The commissure opening is disposed between adjacent outer frame cells and extends radially inwards from the plurality of interconnected angled struts and the vertical struts while the frame is in the radially compressed configuration.

Certain examples concern a method for implanting a prosthetic heart valve, comprising plastically deforming a portion of a medical assembly having an annular frame while the annular frame is in a radially expanded configuration, heat setting the plastically deformed portion of the medical assembly, compressing the medical assembly from the radially expanded configuration to a radially compressed configuration, and releasing the medical assembly from the radially compressed configuration to a radially expanded configuration.

Certain examples concern a medical assembly, comprising a frame comprising a first end, a second end, a central longitudinal axis extending from the first end to the second end, a plurality of interconnected non-actuated struts, and at least one actuated strut extending parallel to the longitudinal axis and coupled to one or more non-actuated struts at a first strut end. The medical assembly also includes an actuator configured to radially expand and radially contract the frame. The actuated strut comprises an axially-extending bore that receives a component of the actuator and a plurality of apertures disposed between the first strut end and a second strut end of the actuated strut.

Certain examples concern a prosthetic heart valve. The prosthetic heart valve comprises a radially expandable frame comprising a first end portion, a second end portion, a longitudinal axis extending between the first end portion and the second end portion, a first post extending along the longitudinal axis, and a second post axially aligned with the first post. The frame is radially movable between a radially compressed state and a radially expanded state. The prosthetic heart valve also comprises a valvular structure disposed within the frame and configured to regulate the flow of blood through the frame and an actuation assembly operatively coupled to the frame, comprising an actuator. The first post is cantilevered and comprises a bore extending axially through the post configured to receive the actuator, a first strut, and a second strut spaced laterally apart from the first strut to define an aperture extending radially through the first post.

Certain examples concern a prosthetic heart valve comprising a radially expandable frame comprising a first end portion, a second end portion, a longitudinal axis extending between the first end portion and the second end portion, a first post extending along the longitudinal axis, and a second post axially spaced apart from first post. The frame is radially expandable between a radially compressed state and a radially expanded state. The prosthetic heart valve also comprises a valvular structure disposed within the frame and configured to regulate the flow of blood through the frame and an actuation assembly operatively coupled to the frame. The actuation assembly comprises an actuator, a stopper, and a nut. The first post is configured to elastically deform between a first state and a second state when the actuator imparts a force on the first post.

Certain examples concern a medical assembly comprising a radially expandable frame having a first end portion, a second end portion, a longitudinal axis extending between the first end portion and the second end portion, a first post extending along the longitudinal axis, and a second post extending along the longitudinal axis. The frame is radially expandable between a radially compressed state and a radially expanded state. The medical assembly also comprises an actuator extending from the first post to the second post. The first post comprises a first window configured to change shape as the frame moves between the radially compressed state and the radially expanded state.

Certain examples concern a prosthetic heart valve comprising a radially expandable frame having a first end portion, a second end portion, a longitudinal axis extending between the first end portion and the second end portion, an axially compressible first post extending along the longitudinal axis and movable between an axially extended configuration and an axially compressed configuration, and a second post axially aligned with the first post. The prosthetic heart valve also comprises a valvular structure disposed within the frame configured to regulate the flow of blood through the frame and an actuation assembly operatively coupled to the frame. The actuation assembly comprises an actuator extending between the first post and the second post, a stopper, and a nut. The frame is radially movable from a radially compressed configuration to a radially expanded configuration and from a radially expanded configuration to a radially compressed configuration by rotating the actuators, wherein when the frame is in the radially expanded configuration, the first post is in the axially extended configuration, and wherein when the frame is in the radially compressed configuration, the first post is in the axially compressed configuration.

Certain examples concern a frame for a medical assembly comprising a first end portion, a second end portion, a longitudinal axis extending between the first end portion and the second end portion, a first post extending along the longitudinal axis, and a second post extending along the longitudinal axis and axially aligned with the first post. The frame is radially expandable between a radially compressed state and a radially expanded state, and the first post comprises a first window configured to change shape as the frame moves between the radially compressed state and the radially expanded state.

Certain examples concern a prosthetic heart valve, comprising a radially expandable frame comprising a first end portion, a second end portion, a longitudinal axis extending between the first end portion and the second end portion, a first post extending along the longitudinal axis, and a second post spaced axially apart from the first post. The frame is movable between a radially compressed state and a radially expanded state. The prosthetic heart valve also comprises a valvular structure disposed within the frame and configured to regulate the flow of blood through the frame and an actuator operatively coupled to the first post and the second post. The first post comprises a mechanical fuse configured to elastically deform from a first state to a second state when the actuator imparts a force on the first post.

Certain examples concern a method for implanting a prosthetic heart valve, comprising, advancing the prosthetic heart valve, constrained by a component of a delivery apparatus, through the vasculature of the patient to a desired implantation site, deploying the prosthetic valve from a crimped state to a radially compressed state by removing the prosthetic valve from the component of the delivery apparatus, and radially expanding the prosthetic heart valve from the radially compressed state to a radially expanded state by rotating an actuator operatively coupled to a frame of the prosthetic heart valve relative to the frame of the prosthetic heart valve. The prosthetic heart valve comprises a frame including an axially oriented first post configured to deflect between an axially extended state and an axially compressed state. When the prosthetic heart valve is deployed from the crimped state to the radially compressed state, the first post deflects from the axially extended state to the axially compressed state. When the prosthetic heart valve is radially expanded from the radially compressed state to the radially expanded state, the first post deflects from the axially compressed state to the axially extended state.

Certain examples concern a method comprising sterilizing the prosthetic heart valve, the frame, the medical assembly, or the stent of any preceding claim.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 depicts a perspective view of a prosthetic heart valve according to one example, shown in a radially expanded configuration.

FIG. 2 depicts a side view of a portion of the frame of the prosthetic heart valve of FIG. 1.

FIG. 3 depicts a side view of the frame of the prosthetic heart valve of FIG. 1, shown in a radially contracted configuration with no barreling of the frame.

FIG. 4 depicts a side view of the frame of the prosthetic heart valve of FIG. 1, shown in a radially contracted configuration with barreling of the frame.

FIG. 5 depicts a side view an exemplary delivery assembly comprising the prosthetic heart valve of FIG. 1 and an exemplary delivery apparatus, which can be used with any of the prosthetic heart valves disclosed herein.

FIG. 6A depicts a side view of a distal end portion of the delivery assembly of FIG. 5, showing the frame of the prosthetic heart valve deployed from within a delivery capsule of delivery apparatus and in a radially expanded configuration.

FIG. 6B depicts a side view of the distal end portion of the delivery assembly of FIG. 5, showing the frame of the prosthetic heart valve in a radially compressed configuration.

FIG. 6C depicts a side view of the distal end portion of the delivery assembly of FIG. 5, showing a delivery configuration in which the prosthetic heart valve is disposed within the delivery capsule of the delivery apparatus in a radially compressed configuration.

FIG. 7 depicts a side view of a portion of the delivery assembly, showing the prosthetic heart valve retained in the radially compressed configuration by an adjustable loop of the delivery apparatus.

FIG. 8 depicts a side view of a section of a frame for a prosthetic heart valve according to one example, the frame comprising apertures in one vertical strut.

FIG. 9A depicts a side view of a section of a frame for a prosthetic heart valve according to some example.

FIG. 9B depicts a side view of a section of a frame for a prosthetic heart valve according to some examples, the frame comprising a commissure opening.

FIG. 10A depicts an end view of a section of a frame for a prosthetic heart valve, the frame comprising vertical struts projecting radially inwards.

FIG. 10B depicts an end view of a frame section for a prosthetic heart valve, the frame comprising a commissure opening projecting radially inwards.

FIG. 11 depicts a side view of a frame section comprising lateral support members according to one example.

FIG. 12 depicts a side view of a frame section comprising lateral support members according to some example.

FIG. 13 depicts a side view of a section of a frame for a prosthetic heart valve comprising a collapsible aperture according to one example.

FIG. 14 depicts a radially interior side view of a section of the frame shown in FIG. 13 operatively coupled to an actuation assembly and in a radially compressed configuration.

FIG. 15 depicts a radially interior side view of a section of the frame shown in FIG. 13 operatively coupled to an actuation assembly and in a radially expanded configuration.

FIG. 16A is a perspective view of a vertical post with a collapsible aperture according to one example.

FIG. 16B is a side elevation view of the vertical post shown in FIG. 16A in the axially extended state.

FIG. 16C is a side elevation view of the vertical post shown in FIG. 16A in the axially compressed state.

FIG. 17 is a graphical depiction of the correlation between the forces acting on a vertical post according to one example, and the resulting compressive deformation of the vertical post.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved.

Although the operations of some of the disclosed examples are described in a particular, sequential order for convenient presentation, it should be understood that this manner of description encompasses rearrangement, unless a particular ordering is required by specific language set forth herein. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

As used herein, “e.g.” means “for example,” and “i.e.” means “that is.”

As used herein, the term “parallel” refers to an orientation between a component and a reference line that is parallel or substantially parallel, allowing for minor angular orientation or curvature. When discussing the orientation of a component with an elongated geometry, such as a strut or actuator, the orientation of that component is defined by an axis drawn along the component's length (that is, a major axis of the component), through the cross-sectional midpoint of the component. When the component being discussed has a bend or curvature, the axis is drawn through the cross-sectional midpoint of each endpoint of the component along the length of the component. For example, the axis can, in the case of a curved component, be defined by a chord extending between the ends of the component and drawn through the cross-sectional midpoint at each end.

The axis of a component can be substantially parallel to a reference line if only a small angle, such as 10 degrees or less, exists between the component and the reference line. Thus, for example, an axis of a component may be described as extending parallel to a reference line (such as a vertical axis of a frame) if it is parallel to the reference line, or within 10 degrees of parallel to the reference line.

### Introduction to the Disclosed Technology

Disclosed herein are various examples of prosthetic heart valves for implantation in the native vasculature of a patient, such as the native annuluses of the patient's heart (for example, the aortic, pulmonary, mitral, or tricuspid valves). The disclosed prosthetic heart valves can also be implanted within vessels in communication with the heart, including a pulmonary artery (for replacing the function of a diseased pulmonary valve, or the superior vena cava or the inferior vena cava (for replacing the function of a diseased tricuspid valve) or various other veins, arteries, and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For instance, in one example, the disclosed prosthetic valves can be implanted within a docking device implanted within the pulmonary artery for replacing the function of a diseased pulmonary valve, such as disclosed in U.S. Patent Publication No. 2017/0231756, which is incorporated by reference herein. In some examples, the disclosed prosthetic valves can be implanted within a docking device implanted within or at the native mitral valve, such as disclosed in PCT Publication No. WO2020/247907, which is incorporated herein by reference. In some examples, the disclosed prosthetic valves can be implanted within a docking device implanted within the superior or inferior vena cava for replacing the function of a diseased tricuspid valve, such as disclosed in U.S. Patent Publication No. 2019/0000615, which is incorporated herein by reference.

To facilitate implantation within a patient, prosthetic valves disclosed herein can be radially compressible and expandable between a radially compressed state and a radially expanded state. Thus, the prosthetic valves can be crimped on or retained by an implant delivery apparatus in the radially compressed state during delivery, such as by a delivery system catheter or adjustable loop disposed around the compressed prosthetic heart valve. The prosthetic heart valves can then be expanded by an expansion mechanism, such as an actuator, to the radially expanded state once the prosthetic valve reaches the implantation site. The frames can also be locked in the desired state of radial expansion by means of a locking mechanism, thereby preventing further radial expansion or compression of the prosthetic heart valve frame. It is understood that the prosthetic valves disclosed herein may be used with a variety of implant delivery apparatuses and can be implanted via various delivery procedures, examples of which is discussed herein.

Because reduced prosthetic heart valve diameters are generally preferred for case of implantation, and because greater radial compression results in greater axial extension while the prosthetic heart valve is in the radially compressed configuration, it can be advantageous to design the prosthetic heart valve with longer, thinner actuators that support a greater range of axial extension while minimizing the radial profile of the compressed prosthetic heart valve.

The radial compression of the prosthetic heart valves, however, can pose several technical challenges. In particular, the prosthetic heart valve may not radially compress evenly along its axial length, instead radially compressing to a greater degree at either axial end, and to a lesser degree towards the axial center of the prosthetic heart valve. This can result in the prosthetic heart valve frame assuming a “barreled” shape while the prosthetic heart valve is in the radially compressed configuration. This barreled shape, in turn, can apply stress on various components of the prosthetic heart valve frame, such as the frame actuators, and result in plastic deformation and/or buckling of the actuators or other frame components. This challenge is especially problematic in prosthetic heart valve frames having longer actuators with narrower cross sections.

Due to the movement of the frame between the crimped state to the functional state (and vice versa), there is a need for frames for prosthetic heart valves that are flexible to allow for the movement and robust to ensure that the frame functions properly both during and after the implantation procedure.

The prosthetic heart valve frame examples disclosed herein include mechanisms to prevent or mitigate the buckling of the actuation members and other frame components. Various examples disclosed herein can include actuated vertical struts with one or more apertures therein to reduce the rigidity of the actuated vertical struts. Other examples can include heat setting one or more frame elements such as an actuated vertical strut or a commissure window to project either radially inwards or radially outwards from the axis of the prosthetic heart valve frame. Still some examples can include adding additional struts to distribute the elastic forces of compressed frame components more evenly across the entire frame. Any of these examples may be used solely, or in combination with any number of some examples.

### The Disclosed Technology and Exemplary Embodiments

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

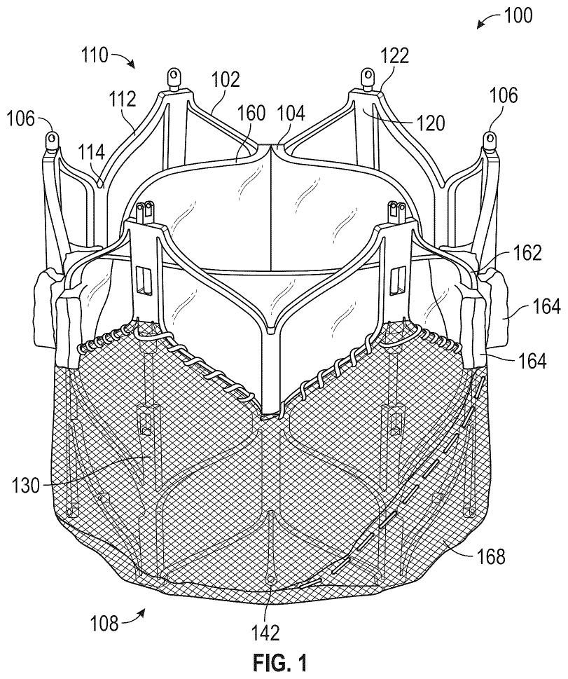

FIG. 1 depicts one example of a prosthetic heart valve which can be radially compressed for delivery through a patient's vasculature and radially expanded to a functional size at a desired implantation location within the patient′ body (for example, the native aortic valve). The prosthetic heart valve 100 (also referred to herein as “the prosthetic valve 100”) comprises a frame 102 and a valvular structure 104.

The frame 102 (which can also be referred to as “a stent” or “a support structure”) can be configured to support the valvular structure 104 and for securing the prosthetic heart valve 100 within a native heart valve and/or within another support structure (for example, an anchoring frame (such as a coil) and/or a previously implanted prosthetic heart valve (that is, in a valve-in-valve procedure). The frame 102 can further comprise one or more actuators 106 configured to radially expand or radially compress the frame 102, as described herein.

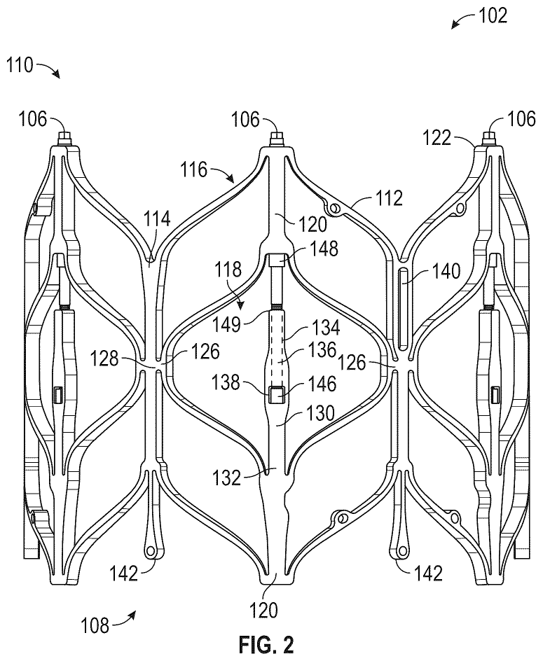

With continued reference to FIGS. 1-2, the frame 102 of the prosthetic heart valve 100 has a first end 108 and a second end 110. In the depicted orientation, the first end 108 of the frame 102 is an inlet end and the second end 110 of the frame 102 is an outlet end. In some examples, the first end 108 of the frame 102 can be the outlet end and the second end 110 of the frame 102 can be the inlet end.

The frame 102 can comprise a plurality of interconnected angled struts 112 and vertical struts 114. In some examples, the angled struts 112 and the vertical struts 114 define a plurality of frame cells. For example, as illustrated in FIG. 2, the angled struts 112 and the vertical struts 114 define a row of six primary cells 116 (which can alternatively be referred to as “outer cells”) and a row of six secondary cells 118 (which can alternatively be referred to as “inner cells) each nested within a respective external cell. The primary cells 116 and the secondary cells 118 can, as illustrated in FIGS. 1 and 2, be connected at their respective axial ends by axial members 120. The primary cells 116 and/or the axial members 120 of the frame 102 can, in some examples, form apices 122 at the first end 108 and the second end 110 of the frame 102.

As illustrated in FIGS. 1 and 2, some of the vertical struts 114 of the frame 102 can be disposed between adjacent pairs of primary cells 116. In some embodiments, the vertical struts 114 can terminate axially inwards from both the first end 108 and the second end 110 of the frame 102. The vertical struts 114 can connect at either axial end to angled struts 112 of the adjacent primary cells 116, such as the two angled struts 112 at each axial end of the vertical struts 114 shown in FIGS. 1 and 2. Accordingly, in some embodiments, the angled struts 112 and the vertical struts 114 can, taken together, define the primary cells 116 of the frame 102, and the primary cells 116 can, as illustrated in FIGS. 1 and 2, have a hexagonal geometry. As shown in FIG. 2, the secondary cells 118 can comprise lateral vertices 126 and can be connected to the vertical struts 114 of the corresponding primary cell 116 by a plurality of lateral members 128, which, in the illustrated embodiments, extend from the lateral vertices 126 of each secondary cell 118 to the nearest corresponding vertical strut 114.

With continued reference to FIG. 2, the frame 102 can also comprise one or more actuated vertical struts 130. The actuated vertical struts 130 can, as shown in FIGS. 1 and 2 be disposed within a secondary cell 118. The actuated vertical struts 130 can be connected at a first end 132 (sometimes called a fixed end 132) to an angled strut 112 or a vertical strut 114 of the secondary cell 118, or to an apex formed by the intersection of two struts 112, 114 of the secondary cell 118. The actuated vertical struts 130 can extend axially from the angled struts 112, the vertical struts 114 or the apex of the secondary cell 118 and can terminate in a second end 134 (called a free end 134 in some examples). The actuated vertical struts 130 can further comprise a bore 136 extending axially from the first end 132 to the second end 134, and configured to receive an actuator, such as actuator 106. In some examples, the actuated vertical strut 130 can further comprise a window 138, configured to receive one or more components of an actuator. Although the actuated vertical struts 130 are described as vertically oriented actuated struts for convenience, it is to be understood that in some examples, the strut may have other nonvertical orientations, such as orientations with a slight angle or curvature relative to an axial axis through the frame.

The frame 102 can further comprise a plurality of leaflet attachment structures. For example, as depicted in FIG. 2, the frame 102 can comprise one or more commissure openings 140 disposed circumferentially between one or more adjacent pairs of the primary cells 116 of the frame 102. The commissure openings 140 can be spaced axially apart from the apices 122 (such as axially inwards) at either the first end 108 or the second end 110 of the frame 102. In the depicted examples, the commissure openings 140 can be bounded on all sides in a “closed” configuration. In some examples, the commissure openings 140 can comprise an open configuration (for example, a U-shaped slot open on one end).

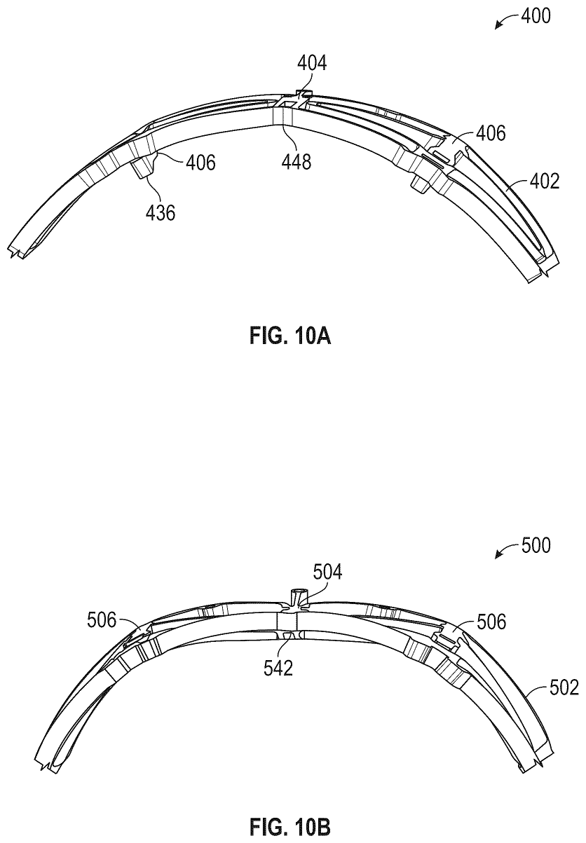

As best illustrated in FIG. 2, the frame 102 can further comprise one or more axially extending suture posts 142. The axially extending suture posts 142 can extend from one or more of the vertical struts 114 as shown in FIGS. 2 and 10. The axially extending suture posts 142 can provide additional locations for affixing the valvular structure 104 or other soft components of the prosthetic heart valve 100.

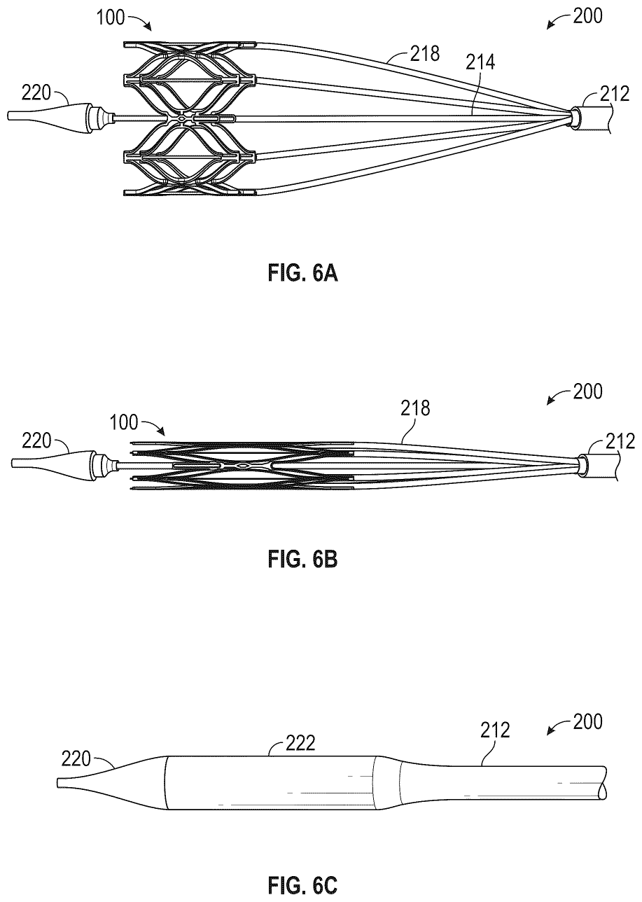

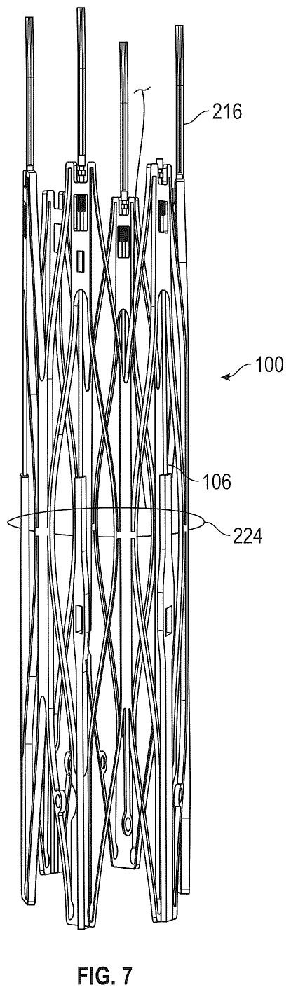

The frame 102 can be configured to move between a plurality of radial configurations, as shown, for example in FIGS. 6A through 7. FIGS. 6B and 7 show a frame 102 in a radially compressed configuration. The depicted configurations are exemplary, and the frame 102 can be expanded or compressed to a lesser or greater extent than depicted. As the frame 102 moves between the various configurations, some of the struts 112, 114 of the frame 102 deflect or pivot relative to each other. For example, the angled struts 112 (which can also be referred to as “diagonal struts”, that is, the non-vertically and non-horizontally oriented struts) deflect relative to the vertically and horizontally oriented struts. In this manner, the frame 102 of the prosthetic heart valve 100 axially elongates when the frame is radially compressed and axially foreshortens when the frame 102 is radially expanded.

While the example prosthetic heart valves described herein include mechanically expandable frames that are expanded by actuators 106, it is to be appreciated that in some examples, different frame expansion mechanisms could be used. For example, self-expanding, partially self-expanding, and balloon expandable frames 102 could be used in place of a mechanically actuated frame as previously described.

Referring again to FIGS. 1-4, the prosthetic heart valve 100 can comprise one or more actuators 106. The actuators 106 are mounted to and spaced circumferentially around the frame 102. In the example illustrated in FIG. 1, the prosthetic heart valve 100 comprises six actuators 106, but it is to be understood that in some examples, fewer actuators (for example, 1-5 actuators) or more actuators (for example, 7-15 actuators) may be used instead. The actuators 106 are configured to, among other things, radially expand and/or radially compress the frame 102.

The actuators 106 can have various forms. For example, in some instances, the actuators 106 can be a rod or shaft. In such instances, the actuators 106 can be formed as separated components from the frame 102, which are then coupled thereto (for example, via welding, adhesive, fasteners, or other means for coupling). Alternatively, the actuators 106 and the frame 102 can be integrally formed as a unitary structure (for example, by forming the frame and actuators from a tube). In some instances, the actuators 106 can be a cable, wire, cord, suture, or other relatively flexible material (that is, compared to a shaft or rod). In such instances, the flexible actuator 106 can be coupled to the frame 102 by tying or looping the actuators 106 around the struts 112, 114 of the frame 102 and/or by coupling the actuator 106 to the frame 102 via a fastener (for example, a grommet), adhesive, and/or other means for coupling.

In some examples, the actuators 106 can be configured for rotational actuation. For example, an actuator 106 may comprise external threads along one or more portions of the actuator 106 (for example, similar to a bolt or screw). As illustrated in FIG. 2, the actuators 106 can comprise a lead screw 144, a nut 146, and a stopper 148. A first end portion of the actuator can be coupled to a first portion (for example, an inlet end portion) of the frame (for example, via the head of the screw) such that the actuator 106 can rotate relative to the first portion of the frame but is axially fixed thereto. In this manner, rotating the actuator 106 in a first direction (for example, clockwise) relative to the frame 102 results in radial expansion of the frame 102 as the first end portion of the frame 102 and the second end portion of the frame move axially toward each other along the threads of the actuator 106. Likewise, rotating the actuator 106 in a second direction (for example, counterclockwise) relative to the frame 102 results in radial compression of the frame 102 as the first end portion of the frame and the second end portion of the frame 102 move axially away from each other along the threads of the actuator 106.

In some examples, the actuators can be configured for linear actuation. In such instances, the actuators 106 comprise fixed end portions fixedly coupled to one portion of the frame (for example, the first end portion) and free end portions movably coupled to another portion of the frame (for example, the second end portion). For example, the fixed end portions of the actuators 106 can be coupled to and/or extend axially from the actuated vertical struts 130 at the inlet end portion of the frame 102, across the primary and second cells and through a lumen traversing the actuated vertical struts 130 at the outlet end portion of the frame 102. The actuator 106 can be used to expand the frame 102 by pulling the actuator 106 toward the outlet end portion of the frame while applying an opposing force on the apices of the outlet end portion of the frame (for example, with a delivery apparatus). These axially-opposing forces together apply a compressive force to the frame and result in radial expansion of the frame. The frame can be radially compressed by reducing tension on the actuators and allowing the elastic properties of the frame to radially compress the frame to its neutral or resting state and/or by an external radially inward force (for example, a crimping device and/or native anatomy within a patient's body).

Each of the actuators can be configured to form a releasable connection with one or more respective actuation shafts of a delivery apparatus. This releasable connection can, for example, include a threaded connection, a plurality of interlocking shafts, and other means of forming a releasable connection. Several examples of releasable connections between the actuators and a delivery apparatus are described herein.

The frame 102 optionally may include a locking mechanism configured to retain the frame 102 in the expanded configuration after the prosthetic heart valve has been radially expanded to the desired diameter. The frame 102 can be configured to freely move between various radially expanded/compressed configurations so long as the locking mechanism is disengaged. When the frame 102 is radially expanded to a desired operational diameter, the locking mechanism can be engaged to prevent further radial expansion and/or contraction of the frame 102.

In some examples, such as examples configured to include rotationally driven actuators, the locking of the prosthetic heart valve 100 can be accomplished by the actuators 106 and the nut 146. In some examples, however, and especially examples using actuators other than rotationally driven actuators, different locking mechanisms, such as locking mechanisms incorporating retention tabs or locking elements may be used instead. Further details regarding prosthetic heart valves, including locking mechanisms and the ways in which locking mechanisms can be incorporated in prosthetic heart valve frames such as frame 102, actuators for radially expanding and compressing prosthetic valves, various frame constructions and methods for assembling prosthetic valves can be found in U.S. Application Nos. 63/085,947, filed Sep. 30, 2020, 63/179,766, filed Apr. 26, 2021, 63/194,285, filed May 28, 2021, and PCT Application No. PCT/US2021/040789, filed Jul. 8, 2021, which are incorporated by reference herein.

The frame 102 can be made of any of various suitable plastically-expandable materials (for example, stainless steel, etc.) or self-expanding materials (for example, Nitinol) as known in the art. When constructed of a plastically-expandable material, the frame 102 (and thus the valve 100) can be crimped to a radially compressed state on a delivery catheter and then expanded inside a patient by an inflatable balloon or equivalent expansion mechanism. When constructed of a self-expandable material, the frame 102 (and thus the valve 100) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 102) include, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

When the frame 102 is constructed from a plastically-expandable material, the expansion force required to radially expand the frame is provided by the actuators 106. In some examples, the angled struts 112 and the vertical struts 114 of the frame can be sufficiently rigid to maintain the frame 102 in the radially expanded state against a surrounding native annulus without the use of any locking mechanism 150.

When the frame 102 is constructed from a shape-memory material (for example, Nitinol), the frame 102 can be configured to self-expand from a radially compressed state to at least a partially radially expanded state. In such cases, the actuators 106 can be used to assist in radially expanding the frame in cooperation with the inherent resiliency of the shape-memory material that urges the frame toward the radially expanded state. For example, the frame 102 can be self-expandable from a radially compressed state to a partially radially expanded state. After the frame reaches the partially radially expanded state, the actuators 106 can be used to further expand the frame 102 from the partially radially expanded state to a fully radially expanded state. After the frame reaches the fully radially expanded state, the actuators 106 can be used to overexpand the frame and dilate the native annulus in which the prosthetic valve is implanted. One or more locking mechanisms, as described herein, can be used to retain the frame in the overexpand state against the forces of the surrounding annulus.

Returning to FIG. 1, the valvular structure 104 of the prosthetic heart valve 100 can be coupled to the frame 102 (for example, directly and/or indirectly via other components such a sealing skirt). The valvular structure 104 is configured to allow blood flow through the prosthetic heart valve 100 from the first end 108 (that is, the inlet end) to the second end 110 (that is, the outlet end) in an antegrade direction and to block blood from flowing through the prosthetic heart valve 100 from the second end 110 to the first end 108 in a retrograde direction. The valvular structure can include various components including a leaflet assembly comprising two or more leaflets 160. For example, the valvular structure 104 in the illustrated example comprises a leaflet assembly having three leaflets 160. It is to be understood, however, that in some examples, the valvular structure 104 could comprise a different number of leaflets.

The leaflets 160 of the prosthetic heart valve 100 can be made of a flexible material. For example, the leaflets 160 can be made from in whole or part, biological material, bio-compatible synthetic materials, or other such materials. Suitable biological material can include, for example, bovine pericardium, equine pericardium, porcine pericardium, and/or pericardium from other sources.

The leaflets 160 can be arranged to form commissures 162. The commissures 162 can, for example, be mounted to the frame at the commissure windows 140, as illustrated in FIG. 1. For example, each leaflet 160 can have two commissure tabs 164 on opposite sides of the leaflet 160. Each commissure tab 164 can be paired with an adjacent commissure tab 164 of an adjacent leaflet to form a respective commissure 162. Each pair of commissure tabs 164 can be coupled to a corresponding vertical strut 114 at a commissure window 140, such as by sutures or other fastening means. Each commissure 162 can include one or more reinforcing members, such as fabric reinforcing members, that are sutured to the commissure tabs 164 and/or the vertical struts 114 to reinforce the connection between the commissure tabs 164 and the vertical struts 114.

The inlet or cusp edge portions of the leaflets 160 can be coupled to the frame 102 via various techniques and/or mechanisms. For example, the cusp edge portions of the leaflets 160 can be sutured directly to selected angled struts 112 or vertical struts 114 of primary cells 116 located at the first end 108 (that is, inlet end) of the prosthetic heart valve. Alternatively, the cusp edge portions of the leaflets 160 can be sutured to an inner skirt (for example, a fabric skirt, not shown), which in turn can be sutured to selected angled struts 112 or vertical struts 114 of primary cells 116 located at the first end 108 (that is, the inlet end) of the prosthetic heart valve. The inlet portions of the leaflets 160 can also, in some examples, be coupled to the one or more axially extending suture posts 142 extending from selected vertical struts 114.

With continued reference to FIG. 1, the valvular structure 104 can further include an outer skirt or sealing member 166 disposed around the exterior of the frame 102. The outer skirt can be made of any suitable biocompatible and flexible material, including materials suitable for leaflets 160, or synthetic material, such as any suitable biocompatible fabric (for example, polyethylene terephthalate (PET) fabric). The outer skirt 166 can be attached to the frame 102 by means of sutures, fabric, adhesive and/or other means for mounting, and in certain examples can be attached to the angled struts 112 and/or vertical struts 114 of primary cells 116 located at the first end 108 (that is, the inlet end) of the prosthetic heart valve. The outer skirt 166 can be configured to improve the seal between the prosthetic heart valve 100 and the native heart valve in which the prosthetic heart valve has been implanted.

The skirt can be wholly or partly formed of any suitable biological material, synthetic material (for example, any of various polymers), or combinations thereof. In some examples, the skirt can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the skirt can comprise a fabric without interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the skirt can comprise a non-textile or non-fabric material, such as a film made from any of a variety of polymeric materials, such as PTFE. PET, polypropylene, polyamide, polyetheretherketone (PEEK), polyurethane (such as thermoplastic polyurethane (TPU)), etc. In some examples, the skirt can comprise a sponge material or foam, such as polyurethane foam. In some examples, the skirt can comprise natural tissue, such as pericardium (for example, bovine pericardium, porcine pericardium, equine pericardium, or pericardium from other sources).

Further details regarding prosthetic heart valves, including the valvular structure 104 and manner in which the valvular structure 104 can be coupled to the frame 102 of the prosthetic heart valve 100, can be found in U.S. Pat. Nos. 6,730,118, 7,393,360, 7,510,575, 7,993,394, 8,652,202, and 9,393,110, U.S. Publication No. 2018/0325665, and U.S. Application No. 63/138,890, filed Jan. 19, 2021, which are incorporated by reference herein.

The examples of prosthetic heart valves described herein may be configured to be implanted in the vasculature of a patient by a delivery apparatus. A suitable delivery apparatus may comprise an elongated shaft configured to pass through the vasculature of a patient, one or more delivery actuators to manipulate a prosthetic heart valve within the patient's body, and a control mechanism by which a physician may control the actuators. Some examples of a delivery apparatus may further include a restraining mechanism configured to retain the prosthetic heart valve in a compressed configuration.

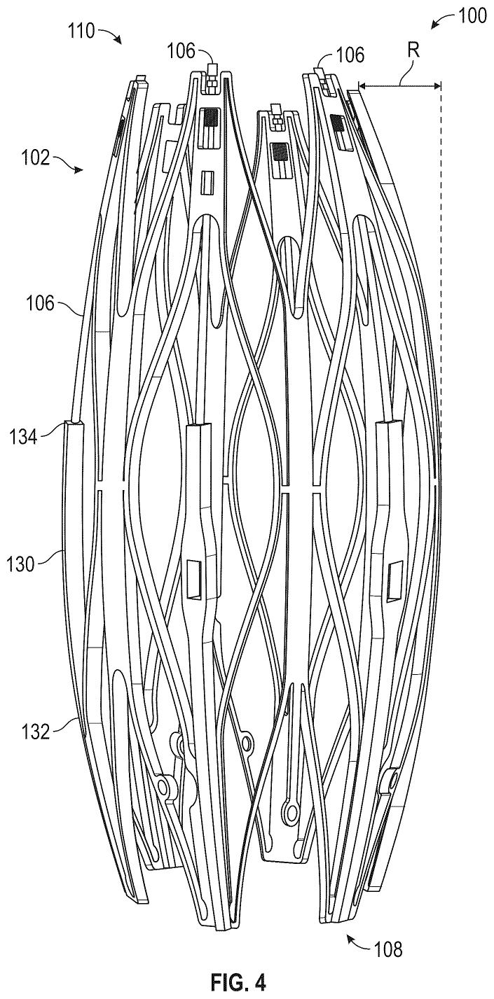

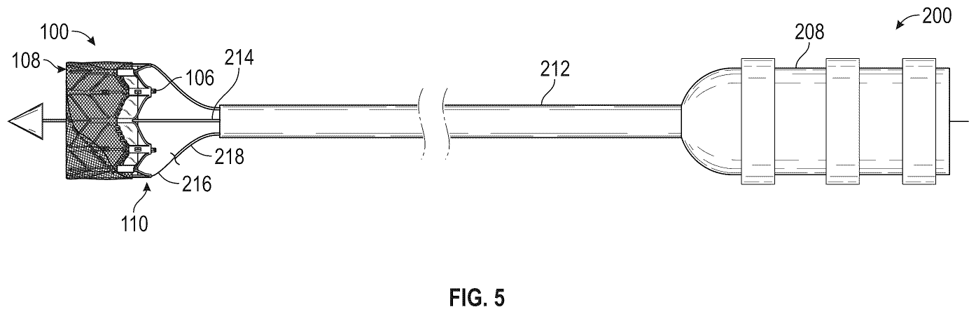

FIG. 5 illustrates a delivery apparatus 200, according to one example, designed to advance a prosthetic heart valve 202 through a patient's vasculature and/or to deliver the prosthetic heart valve 202 to an implantation site (for example, native heart valve) within a patient's body. The prosthetic heart valve 202 can be mounted on, retained within, and/or releasably coupled to a distal end portion of the delivery apparatus 200. The prosthetic valve 202 can represent the prosthetic heart valve 100 previously described herein and illustrated in FIG. 1.

The prosthetic heart valve 202 can include a distal end 204 (which can be the inlet end of the prosthetic heart valve 202, such as when the prosthetic heart valve 202 is configured to replace a defective aortic valve when delivered transfemorally) and a proximal end 206 (which can be the outlet end of the prosthetic heart valve 202, such as when the prosthetic heart valve 202 is configured to replace a defective aortic valve when delivered transfemorally), wherein the proximal end 206 is positioned closer to a handle 208 of the delivery apparatus 200 than the distal end 204, and wherein the distal end 204 is positioned farther from the handle 208 than the proximal end 206. It is to be understood that in some examples, such as when the prosthetic heart valve 202 is implanted in a different location in the vasculature of the patient, the proximal end 206 can alternatively be an inlet end of the prosthetic heart valve 202 and the distal end 204 can be an outlet end of the prosthetic heart valve 202. The prosthetic heart valve 202 can also include one or more actuators 210, extending from the distal end 204 to the proximal end 206, as has been discussed herein.

The delivery apparatus 200 in the illustrated example generally includes the handle 208, a first shaft 212 (an outer shaft in the illustrated example) extending distally from the handle 208, a second shaft 214 (an inner shaft in the illustrated example) extending distally from the handle 208 through the first shaft 212, one or more delivery system actuators 216 extending distally through the outer shaft 212, and one or more support tubes (sometimes called support members) 218 that can extend distally through the outer shaft 212 and can abut the proximal end 206 of the prosthetic heart valve 202. The delivery apparatus 200 can further include a nose cone 220 connected to the distal end portion of the second shaft 214.

Each delivery system actuator 216 can have a distal end connected to an actuator 210 of the prosthetic heart valve 202. Each of the delivery system actuators 216 can extend through a respective support tube 218 and together can define a respective actuator assembly that can extend through the outer shaft 212 to the handle 208. In alternative examples, the delivery system actuators 216 and the support tubes 218 need not be co-axial with respect to each and instead can extend side-by-side through the shaft.

When the prosthetic heart valve includes linear actuators 210, the delivery system actuators 216 and/or the support tubes 218 can be configured to radially expand the prosthetic heart valve 202 by bringing the ends 204, 206 of the prosthetic heart valve 202 closer together (that is, squeezing the prosthetic heart valve 202 axially) thereby axially foreshortening and radially expanding the prosthetic heart valve 202. As one example, the delivery system actuators 216 can be configured to be actuated to provide a proximally directed (for example, pulling) force to the actuators 210 of the prosthetic heart valve 202 while the one or more support tubes 218 can be configured to provide a countervailing distally directed (for example, pushing) force to the proximal end 206 of the prosthetic heart valve 202. The actuators 210, in turn, may transmit the force to the distal end 204 of the prosthetic heart valve 202. In one such example, a physician can pull the delivery system actuators 216 to provide the proximally directed force to the distal end 204 of the prosthetic heart valve 202, while simultaneously gripping, holding, and/or pushing the handle 208 to provide the countervailing distally directed force to the proximal end 206 of the prosthetic heart valve 202.

When the prosthetic heart valve includes rotationally-driven actuators 210, the delivery system actuators 216 can be configured to apply a rotational force to the actuators 210. In such examples, the actuators 210 may have a first threaded end configured to connect with a corresponding threaded end of a delivery system actuator 216. When the delivery system actuator 216 is rotated in a first rotational direction, the actuator 210 can exert an axial force in the proximal direction on the distal end 204 of the prosthetic heart valve 202, thereby axially foreshortening and radially expanding the prosthetic heart valve 202. When the delivery system actuator 216 is rotated in a second rotational direction opposite to the first rotational direction, the actuator 210 can exert an axial force in the distal direction on the distal end 204 of the prosthetic heart valve 202, thereby axially extending and radially contracting the prosthetic heart valve 202. In such an example, a physician can rotationally manipulate the actuators 210 of the prosthetic heart valve 202 to radially expand or contract the prosthetic heart valve 202 to a desired diameter.

As described herein, the delivery system actuators 216 can cooperate with a locking element on the prosthetic heart valve 202 to retain the prosthetic heart valve in a radially expanded state.

Although two pairs of delivery system actuators 216 and support tubes 218 are shown in FIG. 5, it should be understood that the delivery apparatus 200 can include more or less than three delivery system actuators 216 and/or three support tubes 218, in some examples. As just one example, the delivery apparatus 200 can include six delivery system actuators 216 and/or six support tubes 218. In some examples, a greater or fewer number of delivery system actuators 216 and/or support tubes 218 can be present, such as four, five, seven, and/or eight delivery system actuators 216 and/or four, five, seven, and/or eight support tubes 218. In some examples, the delivery apparatus 200 can include equal numbers of delivery system actuators 216 and support tubes 218. However, in some examples, the delivery apparatus 200 can include a different number of delivery system actuators 216 and support tubes 218.

Examples of the delivery apparatus disclosed herein may further include a restraining mechanism configured to retain the prosthetic heart valve in a compressed state. The restraining mechanism may be releasably attached to the prosthetic heart valve while the prosthetic heart valve is being advanced through the vasculature of the patient and/or being positioned at the desired implantation site and may be detached once the prosthetic heart valve has been positioned in the desired location.

In some examples, illustrated in FIG. 6C, the restraining mechanism is a delivery capsule 222 (which may also be referred to as a “sheath”) configured to surround and restrain the prosthetic heart valve in a radially compressed state. The delivery capsule 222 can extend from the distal end of the outer shaft 212 of the delivery apparatus 200, or it can be an integral component of the outer shaft 212. When delivery apparatus 200 advances the prosthetic heart valve 202 to the implantation site, the delivery capsule 222 can be retracted in the proximal direction (that is, towards the handle of the delivery apparatus) to deploy the prosthetic heart valve 202. When the prosthetic heart valve 202 is deployed from the delivery capsule, as shown in FIGS. 6A and 6B, the prosthetic heart valve may be expanded to the partially radially expanded state (FIG. 6B) or to the fully radially expanded state (FIG. 6A).

In lieu of or in addition to a delivery capsule, as illustrated in FIG. 7, the restraining mechanism can include an adjustable loop or lasso 224 circumferentially disposed around the exterior of the prosthetic heart valve 202. The adjustable loop is configured to allow the prosthetic heart valve to expand to the partially radially expanded state (FIG. 4B) or to the fully radially expanded state (FIG. 4A) by introducing slack in the loop 224, allowing the loop 224 to increase in diameter.

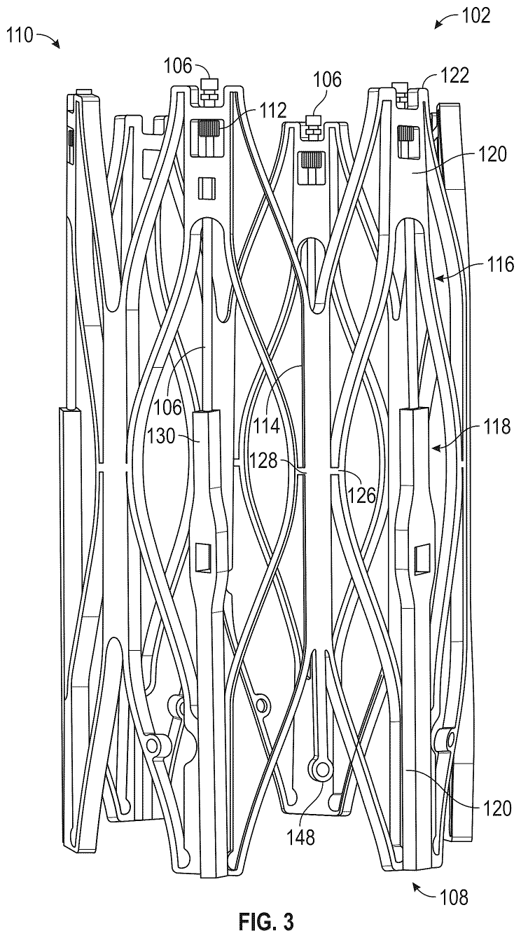

Also disclosed herein are various examples of prosthetic heart valves having frame elements configured to reduce the bending or buckling of the actuators, such as actuators 106. When the prosthetic heart valve 100 is in the radially compressed configuration, the frame 102 can tend to compress more at the first end (sometimes called the distal end) 108 and the second end (sometimes called the proximal end) 110 than at the axial midpoint of the frame, in a phenomenon sometimes known as “barreling”, shown in FIG. 4. Without being limited to any particular theory, it is currently believed that this difference in contraction is due to the higher radial strength of the frame 102 closer to the axial midpoint. The actuated vertical struts 130, in some examples, can be attached to the frame at only the first end 132 (that is, at the fixed end 132), as shown in FIGS. 3 and 4. Thus, the frame 102 may exert a compressive force on the fixed end 132 of the actuated vertical strut 130 as the prosthetic heart valve 100 is radially compressed, while exerting reduced compressive force on the second end 134 (that is, the free end) of the actuated vertical strut 130. This may cause the actuated vertical strut to cantilever out from the frame 102 as the prosthetic heart valve 100 is radially compressed with a radial distance, R, between the second end 134 of the actuated vertical strut 130 and the ends 108, 110 of the frame 102.

As shown in FIG. 4, the actuators 106 can be in contact with the frame 102 at or near the second end (sometimes called the proximal end) 110, and at the second end 134 of the actuated vertical strut 130. Because the second end 134 of the actuated vertical strut 130 may be radially compressed to a lesser degree than either the first end (sometimes called the distal end) 108 or the second end (sometimes called the proximal end) 110 of the frame 102, or the actuated vertical strut 130, the frame 102 can impart a bending moment on the actuators 106, causing the actuators 106 to bow radially outwards relative to the frame 102, as illustrated in FIG. 4. In some cases, this outwards radial bowing of the actuators 106 can cause the actuators 106 to buckle (that is, to plastically deform from a linear configuration). Bowed actuators may subsequently pose several problems during the implantation procedure, particularly during any steps requiring the expansion of the prosthetic heart valve from the radially compressed state to a partially radially expanded state or a fully radially expanded state.

The buckling of actuators can be addressed in several ways in the various examples disclosed herein. In some examples, the frame of the prosthetic heart valve can include actuated vertical struts having one or more apertures. In some examples, the frame of the prosthetic heart valve can include actuated vertical struts that are heat set radially away from the outer circumference of the frame. In yet some examples, the frame of the prosthetic heart valve can include commissure openings that are heat set away from the outer circumference of the frame. In still some examples, the frame can include one or more lateral struts that connect the actuated vertical struts to the cells of the frame. It is to be appreciated that any of these frame elements may be used alone, or in conjunction with any or all of the other frame elements disclosed herein. The various example prosthetic heart valves disclosed herein can, therefore, reduce or eliminate the problems associated with the buckling of the actuators, as will be discussed herein.

In some examples, the actuated vertical strut (such as actuated vertical strut 130 previously described) of a prosthetic heart valve frame (such as frame 102 previously described) can include one or more apertures set in the length of the actuated vertical strut. These apertures can serve to reduce the structural rigidity of the actuated vertical strut (that is, the inclusion of the apertures can reduce the flexural modulus of the actuated vertical strut, increasing its tendency to flex under bending forces). When prosthetic heart valves according to the present disclosure are held in the radially compressed configuration, the barreling of the frame illustrated in FIG. 4 will cause the actuator to impart a radially inward bending force on the free end of the actuated vertical strut. In example prosthetic heart valves having an actuated vertical strut with reduced rigidity (that is, increased flexibility), the bending moment applied to the free end of the actuated vertical strut by the contact between the actuated vertical strut and the actuator may result in a greater radially inwards deflection of the actuated vertical strut. As a result, the radial distance between the free end of the actuated vertical strut and the ends of the frame may be smaller in such examples, and the corresponding bowing of the actuator can be reduced.

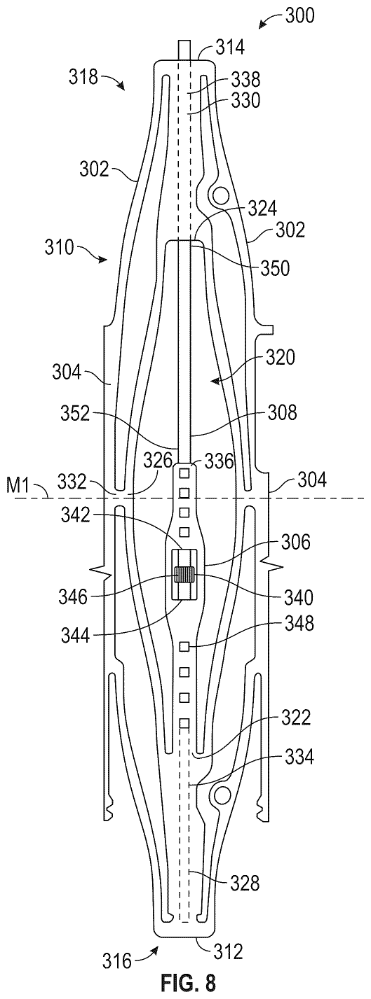

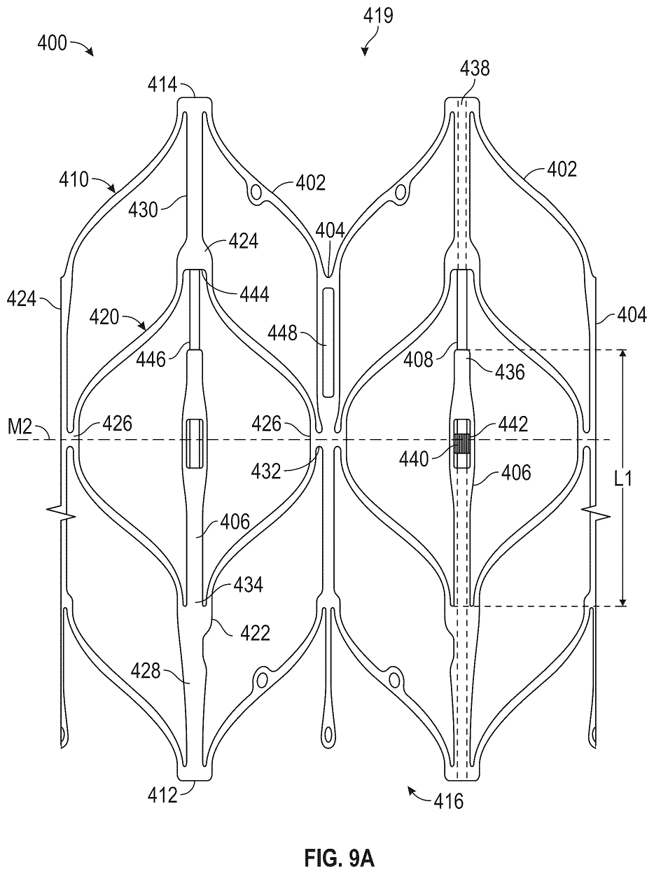

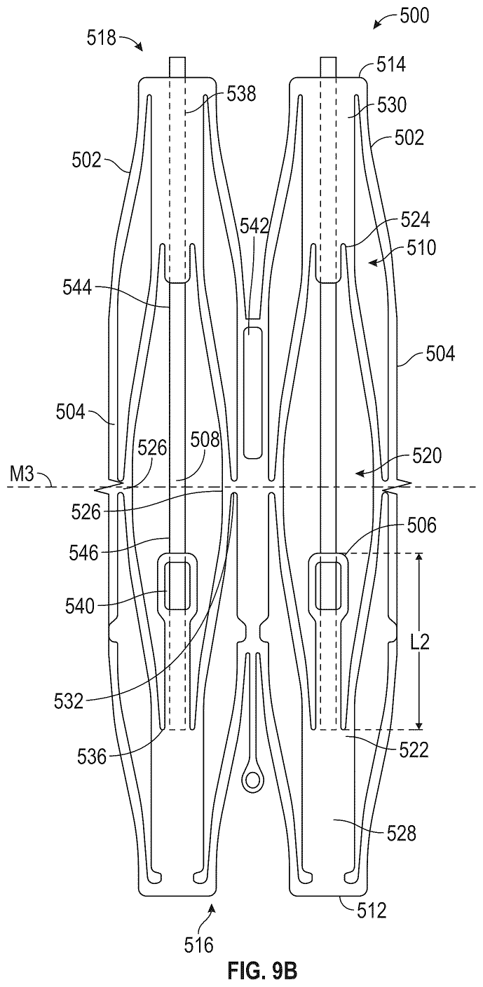

FIG. 8 illustrates a portion of a frame 300 which can include an actuated vertical strut having a plurality of apertures for increased flexibility. As shown in FIG. 8, the frame 300 comprises a plurality of angled struts 302, a plurality of vertical struts 304, and one or more actuated vertical struts 306, and can be configured to receive one or more actuators, such as actuators 308. The frame 300 can comprise a plurality of portions similar or substantially identical to the one shown in FIGS. 9B and 10B arranged adjacent to each other to form an annular frame for a prosthetic heart valve. In some examples, the frame 300 can comprise six outer cells, but in some examples, a greater or lesser number of outer cells such as 4, 5, 7, 8, 9, 10, 11, or 12 outer cells can make up frame 400.

As shown in FIG. 8, the frame 300 can comprise a plurality of interconnected angled struts 302 and vertical struts 304 that form a plurality of outer cells 310 (sometimes called primary cells 310). Each outer cell 310 has an outer distal apex 312 and an outer proximal apex 314. In some examples, such as the one illustrated in FIG. 8, one or more outer distal apices 312 may define an inlet end 316 of the frame 300 and one or more outer proximal apices may define an outlet end 318 of the frame 300. It is to be understood that in some examples, however, the outer distal apices 312 may define the outlet end 318 of the frame 300 and the outer proximal apices 314 may define the inlet end 316 of the frame. The outer cells 310 can also comprise two vertical struts 304, and each outer cell 310 can be connected to two adjacent outer cells 310 along shared vertical struts 304 to form the frame 300.

With continued reference to FIG. 8, the plurality of angled struts 302 and vertical struts 304 can also form an inner cell 320 (sometimes called a secondary cell 320). The inner cell 320 can have an inner distal apex 322 and an inner proximal apex 324, as well as two medial vertices 326. In some examples, such as that shown in FIG. 8, a first axial member 328 can extend from the inner distal apex 322 of the inner cell 320 to the outer distal apex 312 of the corresponding outer cell 310, and a second axial member 330 can extend from the inner proximal apex 324 of the inner cell 320 to the outer proximal apex 314 of the corresponding outer cell 310 to connect the inner cells 320 to the corresponding outer cells 310. In some examples, lateral members 332 can extend from the medial vertices 326 of the inner cells to the vertical struts 304 of the corresponding outer cells. While FIG. 8 shows an inner cell connected to a corresponding outer cell by two axial members and two lateral members, it is to be understood that one or more of these connecting members may, in alternative examples, be absent from the frame 300.

As shown in FIG. 8, the frame 300 can also include an actuated vertical strut 306. The actuated vertical strut 306 can have a fixed end 334 and a free end 336. The actuated vertical strut 306 can attach at the fixed end 334 to the inner distal apex 322 of a secondary cell 320 and can extend axially from the inner distal apex 322 towards the inner proximal apex 324 of the secondary cell 320 while leaving the free end 336 unattached to any other component of the frame. For example, the actuated vertical strut can be cantilevered. In some examples, such as that shown in FIG. 8, the free end 336 of the actuated vertical strut 306 can extend past an axial midpoint M1 of the secondary cell 320 when the frame 300 is in the radially compressed or partially radially expanded configuration. It is to be appreciated, however, that in alternative examples, the actuated vertical strut 306 can be shorter than shown in FIG. 8, such that the free end 336 of the actuated vertical strut 306 is axially aligned with the axial midpoint M1 of the inner cell 320, or stops axially short of the axial midpoint M1 of the inner cell 320. It is also to be appreciated that in some examples, the actuated vertical strut 306 can be longer than shown in FIG. 8. Although the actuated struts 306 are described as vertically oriented actuated struts for convenience, it is to be understood that in some examples, the strut may have other nonvertical orientations, such as orientations with a slight angle or curvature relative to an axial axis through the frame.

In some examples, as shown in FIG. 8, a channel 338 can extend through the first axial member 328, the second axial member 330 and the actuated vertical strut 306. The channel 338 can be configured to admit the actuator 308, which can extend from the inlet end 316 of the frame 300 towards the outlet end 318 of the frame 300. The actuator 308 can be configured, as previously discussed, to draw the inlet end 316 and the outlet end 318 of the frame 300 closer together, thereby axially foreshortening and radially expanding (that is, from a radially compressed configuration to a partially radially expanded configuration or from a partially radially expanded configuration to a fully radially expanded configuration) the frame 300. Likewise, the actuator 308 can be configured to push the inlet end 316 and the outlet end 318 of the frame 300 further apart, thereby axially extending and radially contracting (that is, from a fully radially expanded configuration to a partially radially expanded configuration or from a partially radially expanded configuration to a radially compressed configuration) the frame 300. In some examples, the channel 338 can also be configured to admit a delivery system actuator, such as delivery system actuator 216.

In examples in which the prosthetic heart valve includes actuators 308 configured for rotatable actuation, the actuated vertical strut 306 can further comprise a window 340. The window 340 can have a proximal end 342 and a distal end 344, and can be configured to accommodate a component of the actuator 308, such as an actuation nut 346, which rests within the window 340 and is threadably attached to a portion of the actuator 308. In such examples, because the actuation nut 346 cannot move in the proximal direction (that is, towards the outlet end 318 of the frame 300) past the proximal end 342 or in the distal direction (that is, towards the inlet end 316 of the frame 300) past the distal end 344 of the window 340, and because the nut is threadably attached to the actuator 308, the nut may limit the axial range of motion of the actuator 308.

With continued reference to FIG. 8, the actuated vertical strut 306 can also include one or more apertures 348 disposed between the fixed end 334 and the free end 336. The apertures 348 can reduce the flexural rigidity of the actuated vertical strut 306, causing the axially extending vertical strut to more easily flex radially inwards or outwards from a neutral position. Because the actuated vertical strut 306 having one or more apertures 348 is less rigid than an actuated vertical strut omitting the apertures, but can be otherwise identical, the actuated vertical strut 306 can deflect radially inwards to a greater degree when the frame 300 is in the radially compressed configuration. As a result, in such examples, the radial displacement between the free end 336 of the actuated vertical strut 306 and the ends 316, 318 of the frame 300 can be less than such a radial displacement in a frame having an actuated vertical strut omitting such apertures (that is, an actuated vertical strut with greater stiffness). In turn, this can reduce the radial displacement (shown in FIG. 4 as R) between an end portion 350 and a central portion 352 of the actuator 308 when the frame 300 is in a radially compressed configuration. This can reduce the degree of bending of the actuator 308 when the frame 300 is in a radially compressed configuration, and can reduce the likelihood of plastic deformation and/or buckling of the actuator 308.

In some examples, elements of a frame (such as frame 102) can be plastically deformed or heat set in a deformed configuration suitable for protecting any actuators connected to the frame from bending and/or buckling when the frame is in the compressed configuration. In some examples, the actuated vertical struts can be plastically deformed or heat set to extend radially inwards from the other struts of the frame when the frame is in the radially expanded configuration, resulting in a smaller radial displacement between the actuated vertical struts and the ends of the frame when the frame is in the radially compressed configuration. In some examples, frame struts having commissure openings can be plastically deformed or heat set to extend radially inwards from adjacent struts of the frame when the frame is in the radially expanded configuration, tending to exert force on adjacent actuated struts and reducing the radial displacement between the actuated vertical struts and the ends of the frame when the frame is in the radially compressed configuration.

FIGS. 9A and 10A illustrate sections of one example of a frame 400 having heat set or plastically deformed components, in a radially compressed or partially radially expanded configuration. As shown in FIG. 9A, the frame 400 comprises a plurality of angled struts 402, a plurality of vertical struts 404, and one or more actuated vertical struts 406, and can be configured to receive one or more actuators, such as actuators 408. The frame 400 can comprise a plurality of portions similar or substantially identical to the one shown in FIGS. 9A and 10A arranged adjacent to each other to form an annular frame for a prosthetic heart valve.

As shown in FIG. 9A, the plurality of angled struts 402 and the plurality of vertical struts 404 can form a plurality of outer cells 410 (sometimes called primary cells 410). The outer cells 410 can each have an outer distal apex 412 and an outer proximal apex 414. In some examples, the outer distal apices 412 of the plurality of outer cells 410 can define an inlet end 416 of the frame 400 and the outer proximal apices 414 of the plurality of outer cells 410 can define an outlet end 418 of the frame 400. It is to be understood, however, that in some examples, the outer distal apices 412 may define the outlet end 418 of the frame 400 and the outer proximal apices 414 can define an inlet end 416 of the frame 400. As shown in FIG. 9, each outer cell 410 can be connected to an adjacent outer cell 410 along a vertical strut 404. In some examples, each outer cell 410 can be connected in this way to two adjacent outer cells and arranged in a circular formation to form an annular frame 400. In some examples, the frame 400 can comprise six outer cells, but in some examples, a greater or lesser number of outer cells such as 4, 5, 7, 8, 9, 10, 11, or 12 outer cells can make up frame 400.

With continued reference to FIG. 9A, the angular struts 402 can also form a plurality of inner cells 420 (sometimes called secondary cells 420). The inner cells 420 can each have an inner distal apex 422 and an inner proximal apex 424, as well as two medial vertices 426. Each inner cell 420 can be disposed within a corresponding outer cell 410, as illustrated in FIG. 9A. In some examples, such as that shown in FIG. 9A, a first axial member 428 can extend from the inner distal apex 422 of the inner cell 420 to the outer distal apex 412 of the corresponding outer cell 410, and a second axial member 430 can extend from the inner proximal apex 424 of the inner cell 420 to the outer proximal apex 414 of the corresponding outer cell 410 to connect the inner cells 420 to the corresponding outer cells 410. In some examples, lateral members 432 can extend from the medial vertices 426 of the inner cells to the vertical struts 404 of the corresponding outer cells. While FIG. 9A shows an inner cell connected to a corresponding outer cell by two axial members and two lateral members, it is to be understood that one or more of these connecting members may, in alternative examples, be absent from the frame 400.

As shown in FIG. 9A, the actuated vertical strut 406 can have a first end (sometimes called a fixed end) 434 and a second end (sometimes called a free end) 436. The actuated vertical strut 406 can attach at the fixed end 436 to the inner distal apex 422 of an inner cell 420, and can extend axially from the inner distal apex 422 towards the inner proximal apex 424 of the inner cell 420. For example, the actuated vertical strut can be cantilevered. In some examples, such as that shown in FIG. 9A, the length L1 of the actuated vertical strut 406 is such that the free end 436 can extend past an axial midpoint M2 of the inner cell 420 when the frame is in the radially compressed or partially radially expanded configuration. It is to be appreciated, however, that in alternative examples, the length L1 of the actuated vertical strut 406 can be shorter than that shown in FIG. 9A, such that the free end 436 terminates at the axial midpoint M2 of the inner cell 420 or between the axial midpoint M2 and the inner distal apex 422 when the frame 400 is in the radially compressed or partially radially expanded configuration. It is also to be appreciated that in some examples, the length L1 of the actuated vertical strut 406 can be longer than that shown in FIG. 9A. Although the actuated struts 406 are described as vertically oriented actuated struts for convenience, it is to be understood that in some examples, the strut may have other nonvertical orientations, such as orientations with a slight angle or curvature relative to an axial axis through the frame.

With continued reference to FIG. 9A, a channel 438 can extend through the second axial member 430 and the actuated vertical strut 406. In some examples, the channel 438 can also extend through the first axial member 428. The channel 438 can be configured to admit at least a portion of the actuator 408, which can extend from the outlet end 418 towards the inlet end 416 of the frame 400. The channel 438 can also be configured to admit at least a portion of a delivery system actuator, such as delivery system actuator 216 shown in FIGS. 5 and 7. In some examples, such as that illustrated in FIG. 9A, the actuated vertical strut 406 can also include a window 440. The window 440 can be configured to accommodate various components of the actuator 408. In some examples, such as those having a rotatably-driven actuator, the window 440 can contain an actuator nut 442 configured to limit the axial range of motion of the actuator 408 as previously discussed.

The frame 400 can also include one or more commissure openings 448, as shown in FIGS. 9A and 10A. The one or more commissure openings can be formed in one or more non-actuated struts, such as vertical struts 404, and can be configured to receive one or more leaflets of a valvular structure. In some examples, such as that illustrated in FIG. 9A, the commissure opening 448 can be closed. Closed commissure openings 448 may advantageously permit a more secure attachment of the leaflets of the valvular structure to the frame 400. However, it is also to be appreciated that the commissure opening 448 may be open, for example at the end of the commissure opening closest to either the inlet end or the outlet end of the frame 400. A commissure opening 448 with an open configuration may, for example, allow an casier attachment of the valvular structure to the frame 400. While FIG. 9A shows the commissure opening 448 formed in a portion of the vertical strut 404 disposed towards the outlet end of the frame 400, it is to be appreciated that in some examples, the commissure opening 448 can be formed a portion of a vertical strut 404 disposed towards the inlet end of the frame 500.

Referring now to FIG. 10A, the actuated vertical strut 406 can extend radially inwards from the other components of the frame 400. As illustrated in FIG. 10A, the free end 436 of the actuated vertical strut 406 can, in this way, be disposed radially inwards of the body of frame 400. In some examples, the radially inwards extension of the actuated vertical strut 406 can be accomplished by plastically deforming the actuated vertical strut 406 radially inwards relative to the frame 400, and then applying a shape setting heat treatment to shape set (sometimes called heat set) the actuated vertical strut in this radially deflected configuration. It is to be appreciated, however, that in some examples, the shape setting heat treatment may be omitted, and the plastic deformation of the actuated vertical strut 406 can be sufficient to retain the actuated vertical strut in a configuration in which it extends radially inwards from the rest of the frame.

In examples of frames having an actuated vertical strut that is heat set inwards relative to the body of the frame, the radially inwards disposition of the free end of the actuated vertical strut while the frame is in the radially expanded configuration can prevent or minimize the bending any actuator connected with the frame when the frame is in the radially compressed configuration. For example, the free end 436 of the actuated vertical strut 406 can have a reduced radial displacement relative to the inlet end 416 and the outlet end 418 of the frame 400 when the frame 400 is in the compressed configuration. In turn, this can reduce the radial displacement between an end portion 444 and a center portion 446 of the actuator 408, illustrated in FIG. 9A, when the frame is in the radially compressed configuration. This reduced radial displacement can reduce the degree of bending of the actuator 408 when frame 400 is in a radially compressed configuration, and can tend to reduce the likelihood of plastic deformation or buckling of the actuator 408.

FIGS. 9B and 10B illustrate a section of some examples frame 500 having heat set or plastically deformed components, in a radially compressed or partially radially expanded configuration. As shown in FIG. 9B, the frame 500 comprises a plurality of angled struts 502, a plurality of vertical struts 504, and one or more actuated vertical struts 506, and can be configured to receive one or more actuators, such as actuators 508. The frame 500 can comprise a plurality of portions similar or substantially identical to the one shown in FIGS. 9B and 10B arranged adjacent to each other to form an annular frame for a prosthetic heart valve.

As shown in FIG. 9B, the plurality of vertical angled struts 502 and the plurality of vertical struts 504 can form a plurality of outer cells 510 (sometimes called primary cells 510). The outer cells 510 can each have an outer distal apex 512 and an outer proximal apex 514. In some examples, the outer distal apices 512 of the plurality of outer cells 510 can define an inlet end 516 of the frame 500 and the outer proximal apices 514 of the plurality of outer cells 510 can define an outlet end 518 of the frame 500. It is to be understood, however, that in some examples, the outer distal apices 512 may define the outlet end 518 of the frame 500 and the outer proximal apices 514 can define an inlet end 516 of the frame 500. As shown in FIG. 9B, each outer cell 510 can be connected to an adjacent outer cell 510 along a vertical strut 504. In some examples, each outer cell 510 can be connected in this way to two adjacent outer cells 510 and arranged in a circular formation to form an annular frame 500. In some examples, the frame 500 can comprise six outer cells, but in some examples, a greater or lesser number of outer cells such as 4, 5, 7, 8, 9, 10, 11, or 12 outer cells can make up frame 400.

With continued reference to FIG. 9B, the angular struts 502 also can form a plurality of inner cells 520 (sometimes called secondary cells 520). The inner cells 520 can each include an inner distal apex 522 and an inner proximal apex 524, as well as two medial vertices 526. Each inner cell 520 can be disposed within a corresponding outer cell 510, as illustrated in FIG. 9B. In some examples, such as the one illustrated in FIG. 9B, a first axial member 528 can extend from the inner distal apex of the inner cell 520 to the outer distal apex 512 of the corresponding outer cell 510, and a second axial member 530 can extend from the inner proximal apex 524 of the inner cell 520 to the outer proximal apex 514 of the corresponding outer cell 510 to connect the inner cells 520 to the corresponding outer cells 510. In some examples, lateral members 532 can extend from the medial vertices 526 of the inner cells to the vertical struts 504 of the corresponding outer cells. While FIG. 9A shows an inner cell connected to a corresponding outer cell by two axial members and two lateral members, it is to be understood that one or more of these connecting members may, in alternative examples, be absent from the frame 500.

As shown in FIG. 9B, the actuated vertical strut 506 can have a first end (sometimes called a fixed end) 534 and a second end (sometimes called a free end) 536. The actuated vertical strut 506 can attach at the fixed end 536 to the inner distal apex 522 of an inner cell 520, and can extend axially from the inner distal apex 522 towards the inner proximal apex 524 of the inner cell 520. For example, the actuated vertical strut can be cantilevered. In some examples, such as that shown in FIG. 9B, the length L2 of the actuated vertical strut 506 is such that the free end 536 can terminate short of the axial midpoint M3 of the inner cell 520 when the frame is in the radially compressed or partially radially expanded configuration. It is to be appreciated, however, that in alternative examples, the length L2 of the actuated vertical strut 506 can be longer than that shown in FIG. 9B, such that the free end 536 terminates at the axial midpoint M3 of the inner cell 520 or terminates between the axial midpoint M3 of the inner cell 520 and the inner proximal apex 524 when the frame 500 is in the radially compressed or partially radially expanded configuration. It is also to be appreciated that in some examples, the length L2 of the actuated vertical strut 506 can be shorter than shown in FIG. 9B. Although the actuated struts 506 are described as vertically oriented actuated struts for convenience, it is to be understood that in some examples, the strut may have other nonvertical orientations, such as orientations with a slight angle or curvature relative to an axial axis through the frame.

With continued reference to FIG. 9A, a channel 538 can extend through the second axial member 530 and the actuated vertical strut 506. In some examples, the channel 538 can also extend through the first axial member 528. The channel 538 can be configured to admit at least a portion of the actuator 508, which can extend from the outlet end 518 towards the inlet end 516 of the frame 500. The channel 538 can also be configured to admit at least a portion of a delivery system actuator, such as delivery system actuator 216 shown in FIGS. 5 and 7. In some examples, such as that illustrated in FIG. 9B, the actuated vertical strut 506 can also include a window 540. The window 540 can be configured to accommodate various components of the actuator 408. In some examples, such as those having a rotatably-driven actuator, the window 540 can contain an actuator nut configured to limit the axial range of motion of the actuator 508 as previously discussed.

As shown in FIGS. 9B and 10B, one or more of the vertical struts can include a commissure opening 542. The commissure opening 542 can be disposed between two adjacent outer cells 510, as shown in FIG. 9B, and can be configured to receive one or more leaflet commissures of a valvular structure attached to frame 500. In some examples, such as that illustrated in FIG. 9B, the commissure opening can be closed. Closed commissure openings 542 may advantageously permit a more secure attachment of the leaflets of the valvular structure to the frame 500. However, it is also to be appreciated that the commissure opening 542 may be open, for example at the end of the commissure opening closest to either the inlet end or the outlet end of the frame 500. A commissure opening 542 with an open configuration may, for example, allow the valvular structure to be more easily attached to the frame 500. While FIG. 10 shows the commissure opening 542 formed in a portion of the vertical strut 504 disposed towards the outlet end of the frame 500, it is to be appreciated that in some examples, the commissure opening 542 can be formed a portion of a vertical strut 504 disposed towards the inlet end of the frame 500.

In some examples, the radially inwards extension of the vertical strut 504 having a commissure window 542 shown in FIG. 10B can be accomplished by plastically deforming the vertical strut 504 radially inwards relative to the frame 500, and then applying a shape setting heat treatment to shape set (sometimes called heat set) the vertical strut 504 in this radially deflected configuration. It is to be appreciated, however, that in some examples, the shape setting heat treatment may be omitted, and the plastic deformation of the vertical strut 504 can be sufficient to retain the vertical strut 504 in a configuration in which it extends radially inwards from the rest of the frame.

With continued reference to FIG. 10B, vertical struts 504 having the commissure opening 542 can extend or bow axially inwards from the outer diameter of frame 500. In examples of frame 500 having a vertical strut 504 extending or bowing axially inwards from the outer diameter of the frame, the adjacent angled struts 502 connected to the vertical struts 504 can be deflected inwards to accommodate the vertical strut 504 having the commissure opening 542. In turn, a portion of this radially inwards deflection can be conveyed to the one or more actuated vertical struts 506. In such examples, the radially inwards deflection of the actuated vertical strut 506 resulting from the radially inwards deflection of the vertical strut 504 can minimize the radial displacement of any actuator connected with the frame. For example, the actuated vertical strut 506 can have a reduced radial displacement relative to the inlet end 516 and the outlet end 518 of the frame 500 when the frame 500 is in the compressed configuration. In turn, this can reduce the radial displacement between an end portion 544 and a distal portion 546 of the actuator 508, illustrated in FIG. 9B. This reduced radial displacement can reduce the degree of bending of the actuator 508 when frame 500 is in a radially compressed configuration and can tend to reduce the likelihood of plastic deformation or buckling of the actuator 508.

In some examples, the actuated vertical struts (for example, the actuated vertical strut 130) of a prosthetic heart valve frame (for example, frame 102) can be mechanically supported at both ends. For example, the frame can include an actuated vertical strut attached at a first end to an apex of an inner cell, and attached to other frame components at a second end by one or more lateral and/or angled struts extending from the second end of the actuated vertical strut to other components of the frame. In alternative examples, the second end of the actuated vertical strut can connect directly to a portion of a cell formed by the interconnected struts of the frame. When the prosthetic heart valves according to such examples are held in the radially compressed configuration, the one or more lateral and/or angled struts extending from the second end of the actuated vertical strut, such as actuated vertical strut, can apply a radially compressive force to the second end of the vertical strut. In turn, this radially compressive force can minimize the radial distance between the second end of the actuated vertical strut and the inlet end and outlet end of the frame (such as inlet end 108 and outlet end 110 of the frame 102). As a result, the corresponding bowing of the actuator, (for example, actuator 106) caused by the radial displacement between the end of the actuated vertical strut when the frame is in the radially compressed configuration.

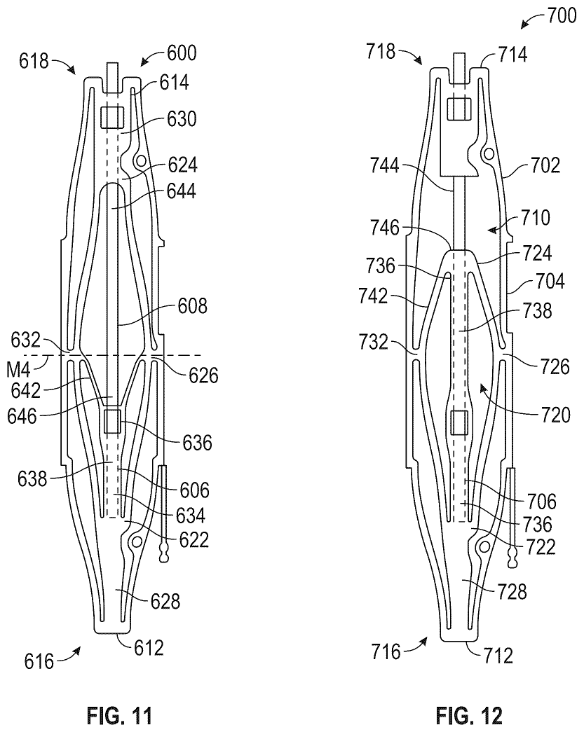

FIG. 11 illustrates one example of a portion of a frame 600 having support for the second end of the actuated strut, in a radially compressed configuration. As shown in FIG. 11, the sections frame 600 can comprise a plurality of angled struts 602, a plurality of vertical struts 604, and one or more actuated vertical struts 606, and can be configured to receive one or more actuators 608. Frame 600 can comprise a plurality of portions similar or substantially identical to the ones shown in FIG. 11 arranged adjacent to each other to form an annular frame for a prosthetic heart valve. In some examples, the frame 600 can comprise six outer cells, but in some examples, a greater or lesser number of outer cells such as 4, 5, 7, 8, 9, 10, 11, or 12 outer cells can make up frame 400.

With continued reference to FIG. 11, the plurality of angled struts 602 and the plurality of vertical struts 604 can form a plurality of outer cells 610 (sometimes called primary cells 610). The outer cells 610 can each have an outer distal apex 612 and an outer proximal apex 614. In some examples, the outer distal apices 612 of the plurality of outer cells 610 can define an inlet end 616 of the frame 600, and the outer proximal apices 614 of the plurality of outer cells 610 can define an outlet end 618 of the frame 600. It is to be understood, however, that in some examples, the outer distal apices 612 may define the outlet end 618 of the frame 600 and the outer proximal apices 614 can define an inlet end 616 of the frame 600. In some examples, each outer cell 610 can be connected in this way to two adjacent outer cells 610 to form an annular frame 600.

As shown in FIG. 11, the angular struts 602 can also form a plurality of inner cells 620 (sometimes called secondary cells 620). The inner cells 620 can each include an inner distal apex 622 and an inner proximal apex 624, as well as two medial vertices 626. Each inner cell 620 can be disposed within a corresponding outer cell 610, as illustrated in FIG. 11. In some examples, such as the one illustrated in FIG. 11, a first axial member 628 can extend from the inner distal apex of the inner cell 620 to the outer distal apex 612 of the corresponding outer cell 610, and a second axial member 630 can extend from the inner proximal apex 624 of the inner cell 620 to the outer proximal apex 614 of the corresponding outer cell 610 to connect the inner cells 620 to the corresponding outer cells 610. In some examples, lateral members 632 can extend from the medial vertices 626 of the inner cells to the vertical struts 604 of the corresponding outer cells. While FIG. 11 shows an inner cell connected to a corresponding outer cell by two axial members and two lateral members, it is to be understood that one or more of these connecting members may, in alternative examples, be absent from the frame 600.

As shown in FIG. 11, the actuated vertical strut 606 can have a first end (sometimes called a fixed end) 634 and a second end (sometimes called a free end) 636. The actuated vertical strut 606 can attach at the first end 634 to the inner distal apex 622 of an inner cell 620, and can extend axially away from the inner distal apex 622 towards the inner proximal apex 624 of the inner cell 620. While FIG. 11 shows that the actuated vertical strut 606 terminates short of the axial midpoint M4 while the frame is in a radially compressed configuration, it is to be appreciated that in alternative examples, the actuated vertical strut 606 can terminate at or past the axial midpoint M4. Although the actuated struts 606 are described as vertically oriented actuated struts for convenience, it is to be understood that in some examples, the strut may have other nonvertical orientations, such as orientations with a slight angle or curvature relative to an axial axis through the frame.

With continued reference to FIG. 11, a channel 638 can extend through the actuated vertical strut 606 and the second axial member 630. The channel 638 can be configured to admit the actuator 608, which can extend from the inlet end 616 end portion of the frame 600 towards the outlet end portion 618 of the frame 600. While FIG. 11 shows an example in which the channel 638 terminates at the inner distal apex 622 of the inner cell 620, it is to be appreciated that in some examples, the channel may extend through the first axial member 628 towards the distal apex 612 of the outer cell. The channel 638 can also be configured to admit components of a delivery system actuator, such as delivery system actuator 216. The actuated vertical strut 606 can also include a window 640. The window 640 can be configured to accommodate various components of the actuator 608. In some examples, such as those having a rotatably-driven actuator, the window 640 can contain an actuator nut configured to limit the axial range of motion of the actuator 608 as previously discussed.

As shown in FIG. 11, the actuated vertical strut 606 can be connected to other components of the frame 600 by one or more lateral support members 642. In examples having an actuated vertical strut 606 that terminates axially short of the inner proximal apex 624 of the free cell (that is, in examples having an actuated vertical strut 606 with a fixed end and a free end), the lateral support members 642 can extend from the second end (that is, free end) 636 of the actuated vertical strut 606 to a component of the frame, such as one or more of the angled struts 604 that define an inner cell 620. In some examples, having an actuated vertical strut 606 that terminates at the inner proximal apex 624 of the inner cell 620, the angled struts 604 that define the inner cell 620 may additionally serve as lateral support members 642. In such examples, the lateral support members 642 may transfer compressive forces from various components of the frame (that is, the angled struts 604 and the actuated vertical struts 606) to the second end 636 of the actuated vertical strut 606. Therefore, when the frame 600 is in a radially compressed state, the lateral support members may tend to apply radially compressive forces on the second end 636 of the actuated vertical strut 606 and thereby cause the actuated vertical strut 606 to more closely conform along its length to the shape of the frame 600 in the compressed configuration. This may reduce the radial displacement between an end portion 644 and a center portion 646 of the actuator 608 caused by the radial compression of the frame 600 and reduce the bending stress on the actuator 608.

In some examples of a section of a frame 700 having support for the free end of the actuated strut, in a radially compressed configuration is shown in FIG. 12. As shown in FIG. 12 the sections frame 700 can comprise a plurality of angled struts 702, a plurality of vertical struts 704, and one or more actuated vertical struts 706, and can be configured to receive one or more actuators 708. Frame 700 can comprise a plurality of portions similar or substantially identical to the ones shown in FIG. 12 arranged adjacent to each other to form an annular frame for a prosthetic heart valve.

As shown in FIG. 12, the plurality of angled struts 702 and the plurality of vertical struts 704 can form a plurality of outer cells 710 (sometimes called primary cells 710). The outer cells 710 can each have an outer distal apex 712 and an outer proximal apex 714. In some examples, the outer distal apices 712 of the plurality of outer cells 710 can define an inlet end 716 of the frame 700, and the outer proximal apices 714 of the plurality of outer cells 710 can define an outlet end 718 of the frame 700. It is to be understood, however, that in some examples, the outer distal apices 712 may define the outlet end 718 of the frame 700 and the outer proximal apices 714 can define an inlet end 716 of the frame 700. In some examples, each outer cell 710 can be connected in this way to two adjacent outer cells 710 to form an annular frame 700. In some examples, the frame 700 can comprise six outer cells, but in some examples, a greater or lesser number of outer cells such as 4, 5, 7, 8, 9, 10, 11, or 12 outer cells can make up frame 400.

In some examples, such as that shown in FIG. 12, the angular struts 702 can also form a plurality of inner cells 720 (sometimes called secondary cells 720). The inner cells 720 can each have an inner distal apex 722 and an inner proximal apex 724, as well as two medial vertices 726. Each inner cell 720 can be disposed within a corresponding outer cell 710 as illustrated in FIG. 12. The inner cell 720 can be connected to the corresponding outer cell by a first axial member 728 extending between the inner distal apex 722 and the outer distal apex 712. In some examples, there may exist no connection between the inner proximal apex 724 and the corresponding outer proximal apex 714, but it is to be understood that in alternative examples, a second axial member may extend between the inner proximal apex 724 and the outer proximal apex 714. In some examples, two lateral members 732 may extend from the medial vertices 726 of the inner cell 720 to the vertical struts 704 of the corresponding outer cell 710.

As shown in FIG. 12, a channel 738 can extend through the actuated vertical strut 706. In some examples, the channel 738 can also extend through the first axial member 728. In examples having a second axial member, the channel 730 can extend through the second axial member. The channel 738 can be configured to admit the actuator 708, which can extend from the inlet end 716 end portion of the frame 700 towards the outlet end portion 718 of the frame 700. While FIG. 11 shows an example in which the channel 738 terminates at the inner distal apex 722 of the inner cell 720, it is to be appreciated that in some examples, the channel may extend further towards the distal apex 712 of the outer cell. The channel 738 can also be configured to admit components of a delivery system actuator, such as delivery system actuator 216. The actuated vertical strut 706 can also include a window 740. The window 740 can be configured to accommodate various components of the actuator 708. In some examples, such as those having a rotatably-driven actuator, the window 740 can contain an actuator nut configured to limit the axial range of motion of the actuator as previously discussed.

With continued reference FIG. 12, the actuated vertical strut 706 can have a first end 734 and a second end 736. The actuated vertical strut 706 can attach at the first end 734 to the inner distal apex 722 of the inner cell 720, and can extend axially away from the inner distal apex 722 and attach to inner proximal apex 724 of the inner cell 720. In this way, the second end 736 of the actuated vertical strut 706 can be coupled to other components of the frame 700, which may apply radially compressive forces on the second end 736 of the actuated vertical strut 706 and thereby cause the actuated vertical strut 706 to more closely conform along its length to the shape of the frame 700 in the compressed configuration. This may reduce the radial displacement between an end portion 744 and a center portion 746 of the actuator 708 caused by the radial compression of the frame 700, and reduce the bending stress on the actuator 708. Although the actuated struts 706 are described as vertically oriented actuated struts for convenience, it is to be understood that in some examples, the strut may have other nonvertical orientations, such as orientations with a slight angle or curvature relative to an axial axis through the frame.

In examples such as those illustrated in FIGS. 11 and 12, the reduced bending stresses on the actuators caused by the radial compression of the frames (that is, frames 600 and 700) can reduce or prevent the buckling of the actuators connected to the frames.

It is to be appreciated that any method of reducing the radial distance (that is, distance R as illustrated in FIG. 4) between an end portion of an actuator and the central portion of the actuator (that is, any method to reduce the bending of the actuator) attached to any of the prosthetic heart valves previously discussed may be used interchangeably. That is, a frame may include an actuated vertical strut with apertures therein, an actuated vertical strut that is heat set or plastically deformed to extend inwards from the outer circumference of the frame, a commissure window heat set or plastically deformed to extend inwards from the outer circumference of the frame, an actuated vertical strut with lateral support members extending therefrom, or any combination thereof.

Also disclosed herein are frames for prosthetic heart valves in which the actuated struts are axial posts (sometimes called vertical posts) with a collapsible window or aperture to facilitate a change in the length of the post along the longitudinal axis of the frame. When stresses are imparted on an axial post including such features, the post can deflect (that is, change shape or elastically deform) to accommodate the actuator and/or to relieve the axial forces acting on the actuator and/or the post. In this way, the degree to which the actuator bends with the frame during the radial compression and/or expansion of the frame can be reduced, and the tendency of the actuator to buckle or bend can be mitigated or prevented.

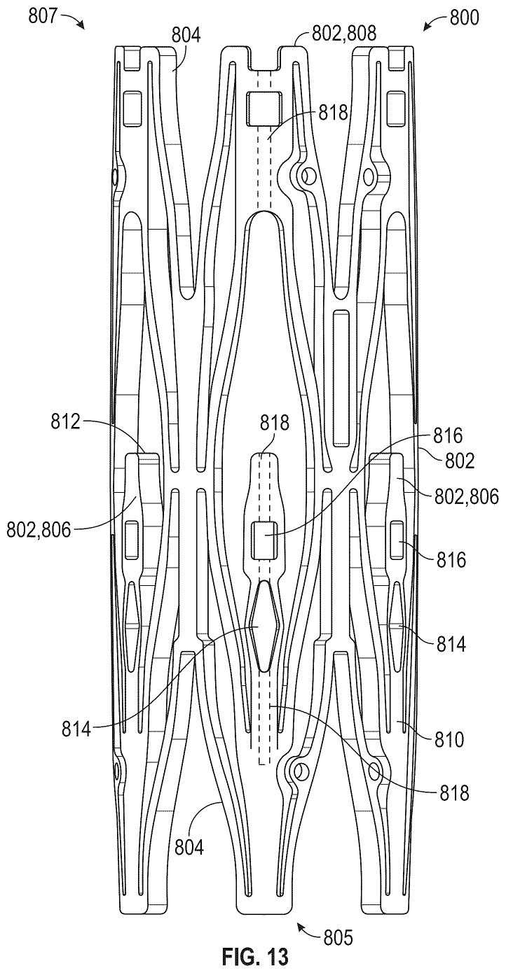

FIG. 13 shows a section of an exemplary prosthetic heart valve frame 800, which includes an axial post with a collapsible window. As shown in FIG. 13, the frame 800 has substantially the same basic configuration as frame 102, as previously described and illustrated in FIGS. 1 and 2, and can generally function in the same way, except for the differences described herein. It is to be understood that, while the frame 800 of FIG. 13 is shown without an attached actuation assembly, actuation assemblies (for example, those previously described in relation to frame 102, and illustrated in FIGS. 1-3) can be used with the frame 800. A prosthetic heart valve can comprise the frame 800 and any of the components described herein for the prosthetic heart valve 100 (for example, leaflets 158, inner and/or outer skirts, connecting member 125, etc.).

Returning to FIG. 13, the frame 800 comprises a plurality of axially oriented posts 802 and a plurality of interconnected angled struts 804 extending between an inflow end 805 and an outflow end 807. Some of the axially oriented posts 802 are arranged in pairs of first posts 806 and second posts 808, which may be circumferentially aligned and axially spaced apart. The first post 806 can comprise a fixed end portion 810 and a free end portion 812 axially spaced apart and can be cantilevered such that the first post 806 is connected to the other frame components at the fixed end portion 810 and left unconnected to the other frame components at the free end portion 812. The first post 806 can further comprise a collapsible first aperture 814 and, optionally, a second aperture 816 (alternatively called a window 816 or a nut window 816). As shown in FIG. 13, the first aperture 814 can be disposed towards the fixed end portion 810 of the first post 806 and the second aperture 816 can be disposed between the first aperture 814 and the free end portion 812 of the first post 806. While FIG. 13 shows a first post 806 having only the collapsible first aperture 814, it is to be understood that in some examples, there can be more than one collapsible aperture or window in the first post, such as a collapsible second aperture or a collapsible third aperture.

With continued reference to FIG. 13, the first post 806 and the second post 808 can comprise an axially oriented bore 818 (sometimes called a channel) configured to receive an actuator of an actuation assembly. The axially oriented bore 818 can extend through the length of the second post 808, and from the free end portion 812 of the first post 806 towards the inflow end 805 of the frame 800. In some instances, the bore 818 comprises threads. In some such instances, the second aperture 816 and a nut (for example, the nut 146 shown in FIG. 2) can be omitted.

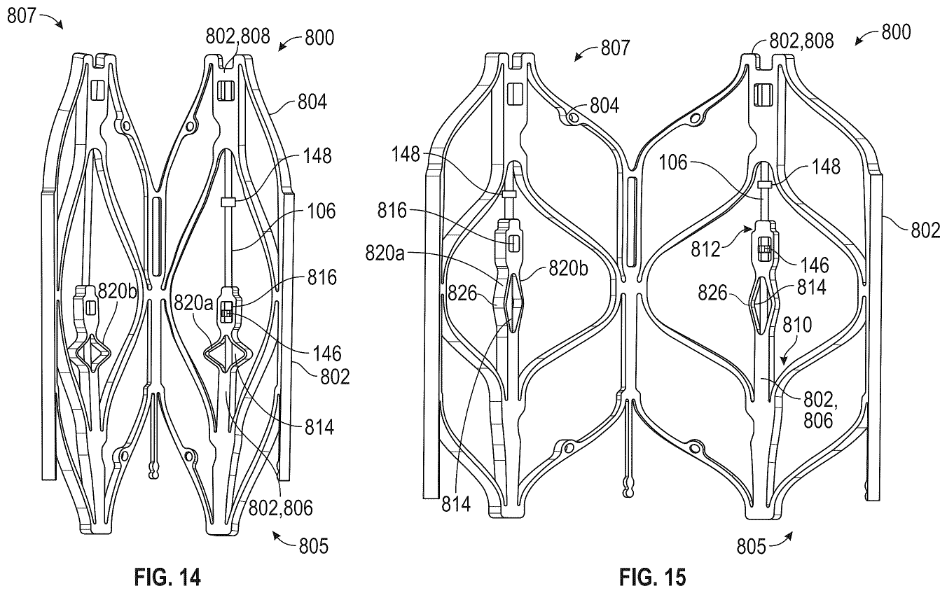

FIGS. 14 and 15 show sections of a frame 800 in the radially compressed and radially expanded state, respectively. As shown in FIGS. 14 and 15, an actuator 106 can extend between the first post 806 and the second post 808. The actuator 106, as discussed herein and illustrated in FIG. 2, can further comprise a lead nut 146 and a stopper 148. The actuator can pass through the bore 818 from the second post 808 to the first post 806. The actuator 106 may also pass through the nut window 816 and the collapsible aperture 814 as illustrated in FIGS. 14 and 15. The lead nut 146 can be disposed in the nut window 816 and can be configured to limit the axial motion of the actuator 106 and to facilitate the radial expansion and compression of the frame 800 as previously discussed. The stopper 148 can be positioned on the actuator 106 between the first post 806 and the second post 808, as shown in FIGS. 14 and 15, and can further limit the axial motion of the actuator 106.

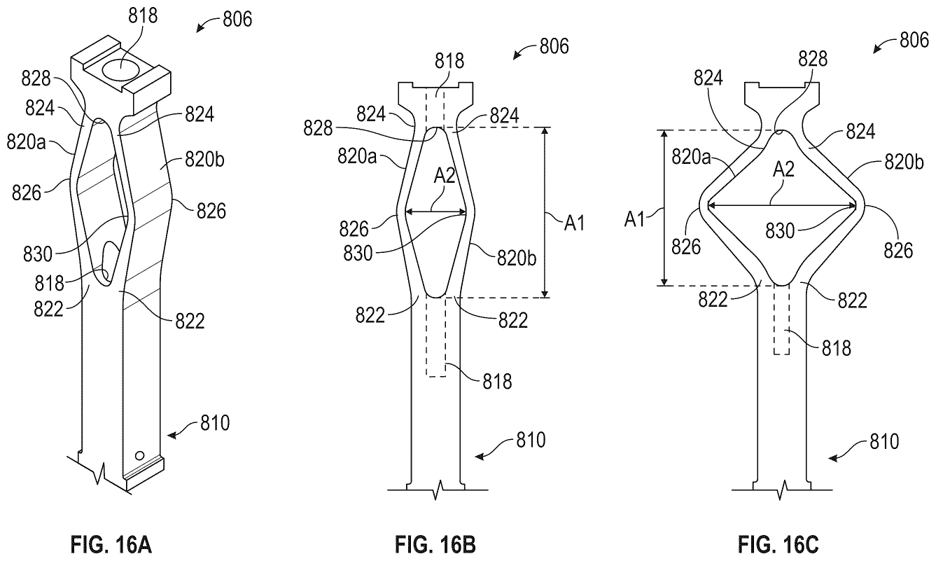

The fixed end portion 810 of the first post 806 with the collapsible aperture 814 is shown in greater detail in FIGS. 16A-16C. As illustrated in FIG. 16A, the collapsible aperture 814 can be defined by a first leg 820a and a second leg 820a, laterally spaced apart from one another. The struts 820a and 820b have a first end portion 822, a second end portion 824, and a joint 826 disposed between the first end portion 822 and the second end portion 824. As illustrated in FIGS. 16A-16C, the first leg 820a and the second leg 820a can be joined at the end portions 822 and 824. In some examples, such as that shown in FIGS. 16A-16C, the joined first leg 820a and second leg 820a form a diamond shaped aperture with a first axis A1 and a second axis A2. The first axis A1 is oriented along the axial length of the first post 806 and extends between two axial vertices 828 formed by the joined end portions 822 and 824 of the legs 820a, 820b. The second axis A2 is oriented transverse to the axial length of the first post 806 and the first axis A1 and extends between two axial vertices 830 located along the joints 826.

As shown in FIG. 16B, the first leg 820a and the second leg 820a can have a variable thickness. In the illustrated example, the first and second struts 820a and 820b can have a greater thickness at the first and second end portions 822, 824, and a lesser thickness at the joint 826. In this way, the first post 806 has a mechanical weak point at the joint 826, which can be used to help ensure that the deflection and/or mechanical deformation of the collapsible aperture 814 occurs primarily at the joints 826.

In some examples, the legs 820a, 820b can have a thickness ranging from 0.15 mm to 0.3 mm at the maximum thickness points along the first and second end portions 822, 824 of each strut. In a specific example, the legs 820a, 820b can have a thickness of 0.2 mm at the maximum thickness points along the first and second end portions 822, 824 of each strut. The legs 820a, 820b can also have a thickness ranging from 0.12 to 0.15 mm at the minimum thickness point at the joint 826 of each strut. In such examples, the legs 820a, 820b can form a collapsible aperture 814 with a height of 3 mm along the first axis A1 and a width of 1.3 mm along the second axis A2, when the window 816 is in an undeflected and/or neutral state (that is, when there are no axially-directed compressive or tensile forces acting on the first post 806).

It will be appreciated by one of ordinary skill in the art that the relative thicknesses of the points of minimum and maximum thickness can be related to one another, and may be selected to control the reaction of the collapsible aperture 814 to axial (that is, tensile or compressive) forces. For example, when the ratio of minimum thickness to maximum thickness is lower, the collapsible aperture 814 may deflect further or under lighter loads, and when the ratio of minimum thickness to maximum thickness is higher, the collapsible aperture 814 can deflect to a lesser degree or require heavier loads to begin deflecting.

In general, when compressive forces act on the first post 806, the legs 820a, 820b forming the collapsible aperture 814 can bend at the joints 826, bringing the axial vertices 828 closer together and pushing the lateral vertices 830 further apart to axially foreshorten the first post 806, as shown in FIG. 16C to an axially compressed state. Conversely, when tensile forces act on the first post 806, or when the compressive force on the first post 806 is removed the legs 820a, 820b forming the collapsible aperture 814 can unbend at the joints 826, bringing the axial vertices 828 further apart and bringing the lateral vertices 830 closer together, as shown in FIG. 16B to axially extend the first post 806 to an axially extended state. Such forces can be caused by the radial expansion and/or contraction of the frame and can be translated to the first post 806 through the actuator 106 as described herein with relation to frames 300, 400, 600, and 700. When the axial length of the first post 806 changes in this way, the shape of the arc that the actuator 106 assumes to accommodate the relative positioning of the first post 806 and the second post 808 can change, and accordingly, the bending stresses on the actuator 106 caused by the change in the curvature of the frame 800 during the deployment of a prosthetic heart valve including the frame 800 can be relieved and/or reduced.

In one example, the prosthetic heart valve including frame 800 can be advanced by a delivery device, such as the delivery device 200 described herein and illustrated in FIGS. 6A through 6C, through the vasculature of the patient to the desired implantation site, and then radially expanded to a desired diameter, as described in greater detail herein.

The prosthetic heart valve including the frame 800 can initially be constrained to a crimped state by a delivery sheath such as the delivery capsule 222 or adjustable loop 224 described herein. While constrained to the crimped state, the frame 800 can have a substantially unbowed shape, and the actuator 106 can be substantially straight along the longitudinal axis of the frame 800. Because the actuator 106 is substantially straight along the longitudinal axis of the frame 800, little or no compressive force is imparted to the first post 806 and the collapsible aperture 814 can remain in the axially extended state.

At the desired implantation site, the prosthetic valve including frame 800 can be deployed from the delivery capsule 222 or the adjustable lasso 224, and the frame 800 can radially expand from the crimped state to a radially compressed state (see FIG. 14). As previously described, the radial expansion can be greater at the axial midsection of the frame 800 than at the inlet end portion 108 or the outlet end portion 109, causing the frame 800 to assume a barreled shape and causing the actuator 106 to bend radially inwards or outwards to accommodate the changing geometry of the frame 800. As the actuator 106 bends radially inwards or outwards, it imparts an axially oriented compressive force to the first post 806. The axially oriented compressive force causes the legs 820a, 820b to bend at the joints 826 to axially collapse the aperture 816 and axially foreshorten the first post 806, as shown in FIGS. 14 and 16C. This in turn can minimize the severity of the bend required to pass the actuator 106 through the bore 818 in both the first post 806 and the second post 808.

The frame 800 can also be mechanically expanded from the radially compressed state to a radially expanded state (see FIG. 15) by rotating the actuator or actuators 106 relative to the first post 806 and the second post 808, in the manner discussed previously in relation to frame 102. As the frame 800 radially expands, diameter of the frame 800 towards the inflow end 805 and the outflow end 109 more closely matches the diameter of the frame 800 towards the axial midpoint of the frame (that is, the frame 800 loses its barreled shape and becomes more cylindrical as it is expanded from the radially compressed state to the radially expanded state). This, in turn, reduces the bending forces on, and thus the severity of the bend of, the actuator 316, allowing the actuator 316 to straighten as the frame 800 radially expands. As the actuator 316 straightens with the radial expansion of the frame 800, the compressive forces imparted to the first post 806 by the actuator 106 are also reduced and the legs 820a, 820b unbend at the joints 826 to axially expand the aperture 816 and axially extend the first post 806, as shown in FIGS. 15 and 16B.

In some circumstances, such as during post ballooning or a valve-in valve procedure, it may also be necessary to expand the diameter of the frame 800 further than can be accomplished solely by the rotation of the actuators 106 relative to the first post 806 and the second post 808. For example, in a valve in valve procedure, a patient has a first prosthetic heart valve pre-installed. In such examples, it may be necessary to replace the valvular structure of the first prosthetic heart valve with the valvular structure of a second prosthetic heart valve. To do this, a second prosthetic heart valve is advanced to the implantation site of the first prosthetic heart valve, and thereafter expanded to the desired diameter. Typically, the desired diameter of the second prosthetic heart valve is large enough to require expansion of the frame of the first prosthetic heart valve to accommodate the second heart valve, such that the frame of the first prosthetic heart valve provides an anchoring site for the second prosthetic heart valve.

In such cases, the frame 800 can be further radially expanded from the radially expanded state to a radially dilated state by applying a force directed radially outwards to the frame 800, which in some examples can be accomplished by inflating an inflatable balloon positioned radially inwards of the frame 800. As the frame 800 is further radially expanded from the radially expanded state to the radially dilated state, the inflow end portion 108 and the outflow end portion 109 are drawn closer together. This in turn causes the ends of the actuator 106 extending between the first post 806 and the second post 808 to draw closer together, compressing and exerting a bending force on the actuator 106. As the actuator 106 is compressed and/or as the actuator 106 begins to bend, it imparts a compressive force on the free end portion 812 of the first post 806, which can cause the legs 820a, 820b to bend at the joints 826 to axially collapse the aperture 816 and axially foreshorten the first post 806 (see FIG. 16C). As described herein, this can relieve the bending forces on the actuator 106 and prevent or mitigate the tendency of the actuator 106 to buckle as the frame 800 is expanded to the radially dilated configuration.

In some examples, the deflection of the legs 820a, 820b (and therefore the deflection of the first post 806) can occur entirely within the elastic region. For example, the legs 820a, 820b may fold along the joints 826 without exceeding the yield stress of the material used. Thus, the components of the first post 806 can, in such examples, experience no plastic deformation during the radial expansion and/or radial compression of the frame. Therefore, when the axial compressive and/or tensile forces imparted to the first post 806 by the actuator 106 are relieved, the legs 820a, 820b and the first post 806 can return to a “neutral” undeflected state. Advantageously, this may allow such an example frame 800 to be radially adjusted multiple times if needed, without plastically deforming one or more components of the first post 806, which may affect further adjustments.

In this way, the compressible aperture 816 can relieve the bending forces experienced by the actuator 106 through the deployment of the prosthetic heart valve including the frame 800. In turn, this mitigates the tendency of the actuator 106 to buckle as the frame is radially expanded and/or compressed to various diameters.

In one specific example, the frame 800 has a diameter of 7 mm while in the radially crimped state within the delivery capsule 222 or the adjustable loop 224, and the first post 806 is in the axially extended state. In this example, when the frame 800 is deployed from the delivery capsule 222 or the adjustable loop 224, the frame 800 expands to a diameter of 13 mm, and the first post 806 deflects from an axially extended state to an axially compressed state. The actuators 106, 316 can then be rotated to expand the frame 800 to a diameter of 27 mm, which causes the first post 806 to deflect from the axially compressed state to the axially extended state. In such an example, the actuators 106, 316 may be unable to radially expand the frame 800 past a diameter of 27 mm, and the frame 800 can be further expanded, for example up to a diameter of 31 mm, by an inflatable balloon, which causes the first post 806 to deflect from the axially extended state to the axially compressed state.

In some examples, the legs 820a, 820b and the compressible aperture 816 formed thereby can be configured to deflect very little while the compressive forces on the first post 806 are below a given threshold, and to deflect greatly while the compressive forces on the first post 806 exceed the given threshold. For example, the collapsible aperture 814 can be configured to function as a mechanical fuse, changing shape as the legs 820a, 820b deflect if the forces on the first post 806 exceed the load threshold.

In this way, the compressible aperture 816 and the first post 806 can be configured to substantially retain their shape while under lower stresses (for example, compressive stresses), such as those which are unlikely to strain the actuator 106 past the yield point and cause plastic deformation, such as that associated with the buckling of the actuators 106, 316. At the same time, the compressible aperture 816 and the first post 806 can also be configured to rapidly deflect under higher stresses to relieve the stresses on the actuator 106 as they approach the yield point and risk plastic deformation and/or catastrophic buckling of the actuator 106.

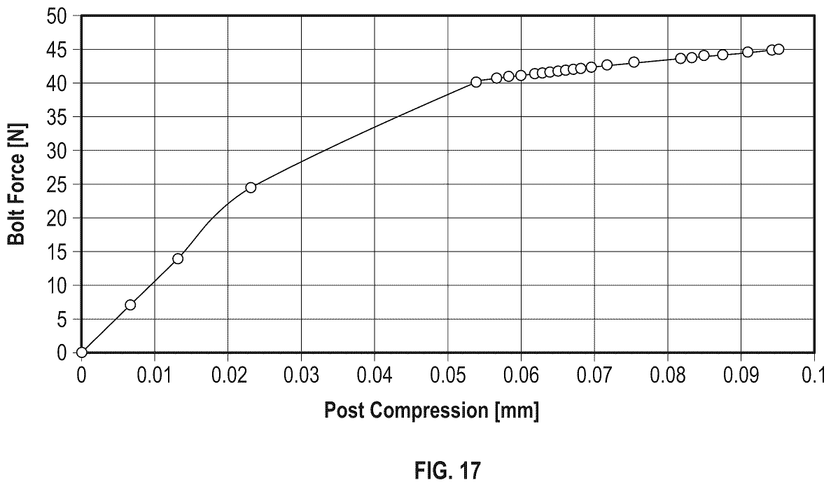

In one particular example, illustrated in FIG. 17, the collapsible aperture 814 is configured to have an actuator force threshold of approximately 40 newtons (40 N). As shown in FIG. 17, in some examples, the collapsible aperture 814 of the first post 806 deflects by less than 0.03 mm when a compressive force of less than or equal to 25 N is applied to the actuator 106 coupled to the frame 800. Similarly, the collapsible aperture 814 of the first post 806 deflects by less than 0.05 mm when a compressive force of less than or equal to 40 N is applied to an actuator 106 coupled to the frame 800. However, the collapsible aperture 814 of the first post 806 deflects by an additional 0.05 mm when a compressive force of 40-45 N is applied to an actuator 106 coupled to the frame 800, for a total deflection approaching 0.1 mm of total deflection at a 45 N compressive force. It should be noted that, for different configurations of the frame 800 and/or the actuator 106 coupled to the frame 800, the actuator force threshold and the corresponding deflection of the collapsible aperture 814 can also be different.

In these ways, the frame 800, having a first post 806 with a collapsible aperture 814 can mitigate or prevent the tendency of the actuator 106 of the prosthetic heart valve assemblies described herein from bending or buckling during the expansion and/or compression of the frame 800, by allowing the first post 806 to relieve the compressive stresses experienced by the actuator 106.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a delivery capsule to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein.

Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire and/or an introducer sheath previously inserted into a patient's vasculature. Moreover, the disclosed delivery approaches are not intended to be limited. Any of the prosthetic valves disclosed herein can be implanted using any of various delivery procedures and delivery devices known in the art.

Advantageously, prosthetic heart valves according to the examples previously discussed can reduce the bending stresses on the attached actuators by reducing the radial displacement between the end portions and the center portions of the actuators. In turn, this may mitigate the tendency of the actuators to bend or buckle during the implantation procedure and reduce resulting impairment to the ability of the prosthetic heart valve to be radially expanded or contracted at the desired implantation site.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples heat/thermal sterilization include steam sterilization and autoclaving. Examples of radiation for use in sterilization include, without limitation, gamma radiation, ultra-violet radiation and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

Example 1. A frame for an implantable device, comprising a first end, a second end, a longitudinal axis extending from the first end to the second end, a plurality of angled struts non-parallel to the longitudinal axis, and a plurality of vertically oriented struts extending parallel to the longitudinal axis and coupled to the angled struts at one or more vertical strut junctions, wherein one or more vertical struts of the plurality of vertically oriented struts includes a fixed end portion connected to a vertical strut junction, a free end portion, and a plurality of apertures disposed between the fixed end portion and the free end portion and configured to increase the flexibility of the one or more vertical struts.

Example 2. The frame of any example herein, particularly example 1, wherein at least one vertical strut of the plurality of vertically oriented struts comprises an axially extending channel extending from a first end of the vertical strut to a second end of the vertical strut configured to receive a first component of an actuator.

Example 3. The frame of any example herein, particularly example 2, wherein the vertical strut additionally comprises a window configured to receive a second component of the actuator.

Example 4. The frame of any example herein, particularly example 3, wherein at least one aperture is positioned between the fixed end of the vertical strut and the window.

Example 5. The frame of any example herein, particularly any of examples 3-4, wherein at least one aperture is positioned between the free end of the vertical strut and the window.

Example 6. The frame of any example herein, particularly any of examples 3-5, wherein the number of apertures between the fixed end of the vertical strut and the window is equal to the number of apertures between the free end of the vertical strut and the window.

Example 7. The frame of any example herein, particularly any of examples 2-6, wherein the actuator comprises a lead screw and a nut and the nut is disposed within the window in the vertical strut.

Example 8. The frame of any example herein, particularly any of examples 2-7, wherein the actuator extends from a first apex at the first end of the frame to a corresponding apex at the second end of the frame.

Example 9. The frame of any example herein, particularly any of examples 2-8, wherein the actuator comprises a locking mechanism.

Example 10. The frame of any example herein, particularly example 2-9, wherein the actuator is configured to attach to an actuator of a prosthetic heart valve delivery apparatus.

Example 11. The frame of any example herein, particularly example 2-10, wherein the actuator is configured to radially expand the frame from a radially compressed configuration to a radially expanded configuration.

Example 12. The frame of any example herein, particularly any of examples 1-11, further comprising a valvular structure attached the frame and having a plurality of leaflets configured to allow blood to flow through the valvular structure from the first end of the frame to the second end of the frame and to prevent blood from flowing through the valvular structure from the second end of the frame to the first end of the frame.

Example 13. The frame of any example herein, particularly any of examples 1-12, further comprising an outer skirt attached to an exterior of the frame.

Example 14. The frame of any example herein, particularly any of examples 1-13, wherein the vertical strut extends radially inwards from the diameter of the frame when the frame is in a radially expanded configuration.

Example 15. The frame of any example herein, particularly any of examples 1-14, wherein the frame comprises at least one stabilization strut extending from the free end of the at least one vertical strut to one of an angled strut or another vertical strut.

Example 16. The frame of any example herein, particularly any of examples 1-15, wherein the frame comprises at least one commissure opening and the commissure opening extends radially inwards from the diameter of the frame when the frame is in the radially expanded configuration.

Example 17. The frame of any example herein, particularly any of examples 1-16, wherein the frame comprises at least one suture post extending axially from a vertical strut.

Example 18. The frame of any example herein, particularly any of examples 1-17, wherein the frame comprises two or more actuators and two or more vertical struts configured to receive a first component of the two or more actuators.

Example 19. The frame of any example herein, particularly example 18, wherein each axially extending vertical strut comprises one or more apertures.

Example 20. The frame of any example herein, particularly any of examples 1-19, wherein the angled struts and the vertical struts are integrally formed as a unitary structure

Example 21. The frame of any example herein, particularly any of examples 1-19, wherein the angled struts and the vertical struts are formed as a non-unitary structure.

Example 22. The frame of any example herein, particularly any of examples 1-21, wherein the plurality of angled struts and the plurality of vertically oriented struts form a plurality of outer cells and a plurality of inner cells nested within the plurality of outer cells, wherein the plurality of inner cells is connected to the plurality of outer cells at one or more vertices of the inner cells.

Example 23. A prosthetic heart valve, comprising a frame having a first end, a second end, a longitudinal axis extending from the first end to the second end, a plurality of angled struts extending transverse to the longitudinal axis, a plurality of vertical struts extending parallel to the longitudinal axis, and a plurality of junctions formed by an intersection of two or more angled struts, vertical struts, or a combination thereof, and a valvular structure having a plurality of leaflets configured to allow blood to flow through the prosthetic heart valve from the first end of the frame to the second end of the frame and to prevent blood from flowing through the prosthetic heart valve from the second end of the frame to the first end of the frame, wherein at least one vertical strut of the plurality of vertical struts has a fixed end and a free end, is attached at the fixed end to one junction of the plurality of junctions, and extends radially inwards from the diameter of the frame with a first radial displacement when the frame is in a radially expanded configuration.

Example 24. The prosthetic heart valve of any example herein, particularly example 23, wherein at least one vertical strut of the plurality of vertical struts comprises an axially-extending channel to receive a first component of an actuator.

Example 25. The prosthetic heart valve of any example herein, particularly example 24, wherein the actuator extends from an apex at the first end of the frame to a corresponding apex at the second end of the frame.

Example 26. The prosthetic heart valve of any example herein, particularly any of examples 24-25, wherein the at least one vertical strut comprises a window configured to receive a second component of the actuator.

Example 27. The prosthetic heart valve of any example herein, particularly any of examples 24-26, wherein the actuator comprises a lead screw and a nut and the nut is disposed within the window of the vertical strut.

Example 28. The prosthetic heart valve of any example herein, particularly any of examples 24-27, wherein the actuator comprises a locking mechanism.

Example 29. The prosthetic heart valve of any example herein, particularly any of examples 23-28, wherein the actuator is configured to attach to a component of a prosthetic heart valve delivery apparatus

Example 30. The prosthetic heart valve of any example herein, particularly any of examples 24-29, wherein the actuator is configured to radially expand the frame from a radially compressed configuration to a radially expanded configuration.

Example 31. The prosthetic heart valve of any example herein, particularly any of examples 23-30, wherein the at least one vertical strut of the plurality of vertical struts extends radially inwards from the diameter of the frame when the prosthetic heart valve is in the radially compressed configuration with a second radial displacement less than the first radial displacement.

Example 32. The prosthetic heart valve of any example herein, particularly any of examples 23-30, wherein the vertical strut does not extend radially inwards relative to the diameter of the frame when the prosthetic heart valve is in the radially compressed configuration.

Example 33. The prosthetic heart valve of any example herein, particularly any of examples 23-32, wherein the at least one vertical strut of the plurality of vertical struts is shape set to radially extend inwards from the diameter of the frame when the prosthetic heart valve is in the radially expanded or partially radially expanded configuration.

Example 34. The prosthetic heart valve of any example herein, particularly any of examples 23-33, wherein the frame comprises at least one stabilization strut extending from the free end of the at least one vertical strut to one of an angled strut or another vertical strut.

Example 35. The prosthetic heart valve of any example herein, particularly any of examples 23-34, wherein the frame comprises at least one commissure opening wherein the commissure opening extends radially inwards the frame when the frame is in the radially expanded configuration.

Example 36. The prosthetic heart valve of any example herein, particularly any of examples 23-35, wherein the at least one vertical strut of the plurality of vertical struts comprises one or more apertures.

Example 37. The prosthetic heart valve of any example herein, particularly any of examples 23-36, wherein the frame comprises at least one suture post extending axially from a vertical strut or an angled strut.

Example 38. The prosthetic heart valve of any example herein, particularly any of examples 23-37, wherein the frame comprises two or more vertical struts configured to receive a first component of two or more actuators.

Example 39. The prosthetic heart valve of any example herein, particularly any of examples 23-38, wherein the frame comprises two or more vertical struts extending radially inwards from the plurality of vertical struts while the prosthetic heart valve is in the radially expanded configuration.

Example 40. The prosthetic heart valve of any example herein, particularly any of examples 23-39, wherein the angled struts and the vertical struts are integrally formed as a unitary structure.

Example 41. The prosthetic heart valve of any example herein, particularly any of examples 23-39, wherein the angled struts and the vertical struts are formed as a non-unitary structure.

Example 42. The frame of any example herein, particularly any of examples 23-41, wherein the plurality of angled struts and the plurality of vertical struts form a plurality of outer cells and a plurality of inner cells nested within the plurality of outer cells, wherein the plurality of inner cells are connected to the plurality of outer cells at one or more vertices of the inner cells.

Example 43. A medical assembly, comprising a radially expandable annular frame having a distal end, a proximal end, a vertical axis extending from the distal end to the proximal end, a plurality of interconnected non-actuated struts, and at least one actuated strut, an actuator configured to radially expand the frame from a radially compressed configuration to a radially expanded configuration; and a commissure opening in at least one of the plurality of non-actuated struts, wherein at least one actuated strut comprises a fixed end connected to one or more of the plurality of non-actuated struts, a free end, and a channel extending from the fixed end to the free end configured to receive a first component of the actuator, and wherein the non-actuated strut having the commissure opening extends radially inwards with a first radial displacement from the adjacent non-actuated struts while the annular frame is in the radially expanded configuration.

Example 44. The medical assembly of any example herein, particularly example 43, wherein the frame further comprises at least one suture post extending axially from an interconnected strut.

Example 45. The medical assembly of any example herein, particularly any of examples 43-44, wherein the actuator extends from a first apex disposed at the distal end of the frame to a second apex radially aligned with the first apex and disposed at the proximal end of the frame.

Example 46. The medical assembly of any example herein, particularly any of examples 43-45, wherein the at least one actuated strut comprises a window configured to receive a second component of the actuator.

Example 47. The medical assembly of any example herein, particularly example 46, wherein the actuator comprises a lead screw and a nut and the nut is disposed within the window of the actuated strut.

Example 48. The medical assembly of any example herein, particularly any of examples 43-47, wherein the actuator comprises a locking mechanism.

Example 49. The medical assembly of any example herein, particularly any of examples 43-48, wherein the non-actuated strut having the commissure opening extends radially inwards from the adjacent non-actuated struts with a second radial displacement that is less than the first radial displacement when the frame is in the radially compressed configuration.

Example 50. The medical assembly of any example herein, particularly any of examples 43-49, wherein the commissure opening does not extend radially inwards from the interconnected struts of the frame when the frame is in the radially compressed configuration.

Example 51. The medical assembly of any example herein, particularly any of examples 43-50, wherein non-actuated struts adjacent to the non-actuated strut having the commissure opening are deflected radially inwards when the frame is in radially expanded configuration.

Example 52. The medical assembly of any example herein, particularly any of examples 43-51, wherein the actuator is not deflected in a radially inward direction relative to the frame when the frame is in the radially compressed configuration.

Example 53. The medical assembly of any example herein, particularly any of examples 43-52, wherein the commissure opening is heat set to extend radially inwards from the interconnected struts of the frame.

Example 54. The medical assembly of any example herein, particularly any of examples 43-53, wherein the actuated strut extends radially inwards from an outer circumference of the frame when the frame is in the radially expanded configuration.

Example 55. The medical assembly of any example herein, particularly any of examples 43-54, wherein the frame comprises at least one stabilization strut extending from the free end of the actuated strut to one of an angled strut or another vertical strut.

Example 56. The medical assembly of any example herein, particularly any of examples 43-55, wherein the actuated strut comprises a plurality of apertures disposed between the fixed end and the free end.

Example 57. The medical assembly of any example herein, particularly any of examples 43-56, wherein the frame comprises two or more actuators and axially extending vertical struts configured to receive the actuators.

Example 58. The medical assembly of any example herein, particularly any of examples 43-57, wherein the medical assembly comprises a valvular structure attached the frame, having a plurality of leaflets configured to allow blood to flow through the medical assembly from the distal end of the frame to the proximal end of the frame and preventing blood from flowing through the medical assembly from the proximal end of the frame to the distal end of the frame.

Example 59. The medical assembly of any example herein, particularly any of examples 43-58, wherein the medical assembly further comprises an outer skirt attached to the frame.

Example 60. The medical assembly of any example herein, particularly any of examples 43-59, wherein the actuator is configured to attach to a component of a prosthetic heart valve delivery apparatus.

Example 61. The medical assembly of any example herein, particularly any of examples 43-60, wherein the medical assembly comprises two or more commissure openings extending inwards from at least a respective angled strut or at least a respective vertical strut.

Example 62. The prosthetic heart valve of any example herein, particularly any of examples 43-61, wherein the angled struts and the vertical struts are integrally formed as a unitary structure.

Example 63. The prosthetic heart valve of any example herein, particularly any of examples 43-62, wherein the angled struts and the vertical struts are formed as a non-unitary structure.

Example 64. The frame of any example herein, particularly any of examples 43-63, wherein interconnected non-actuated struts form a plurality of outer cells and a plurality of inner cells nested within the plurality of outer cells, wherein the plurality of inner cells are connected to the plurality of outer cells at one or more vertices of the inner cells.

Example 65. An implantable stent, comprising a distal end, a proximal end, a longitudinal axis extending between the distal end and the proximal end, plurality of angled struts defining an annular body, a plurality of vertical struts extending parallel to the longitudinal axis, and a plurality of junctions formed by an intersection of two or more angled struts or vertical struts, wherein at least one vertical strut of the plurality of vertical struts comprises a first end attached to a first junction of the plurality of junctions, a body extending from the junction parallel to the longitudinal axis, and a second end at the opposite end of the body from the first end.

Example 66. The stent of any example herein, particularly example 65, further comprising at least one support strut extending from the second end and connecting to at least one of an angular strut or another vertical strut.

Example 67. The stent of any example herein, particularly example 66, wherein the frame comprises two or more lateral support struts extending from the second end to two or more angular struts or other vertical struts.

Example 68. The stent of any example herein, particularly any of examples 66-67, wherein the frame comprises two or more vertical struts having a first end and a second end, connected at the first ends to two or more respective junctions and axially extending from the respective junctions, and connected at the second end to at least one support strut extending from the second end to at least one of an angular strut or another vertical strut.

Example 69. The stent of any example herein, particularly any of examples 65-67, wherein the second end of the at least one vertical strut is located axially between the first junction and an axial midpoint of the stent.

Example 70. The stent of any example herein, particularly any of examples 65-68, wherein an axial midpoint of the stent is located axially between the second end of the at least one vertical strut and the first junction.

Example 71. The stent of any example herein, particularly any of examples 65-68, wherein the second end of the at least one vertical strut terminates at an axial midpoint of the frame.

Example 72. The stent of any example herein, particularly example 65, wherein the second end terminates at a second junction of the plurality of junctions is radially aligned with the first junction.

Example 73. The stent of any example herein, particularly any of examples 65-72, wherein the frame further comprises at least one suture post extending axially from a vertical strut or an angled strut.

Example 74. The stent of any example herein, particularly any of examples 65-72, wherein the at least one vertical strut comprises an axially-extending channel to receive a first component of an actuator.

Example 75. The stent of any example herein, particularly example 73, wherein the at least one vertical strut comprises a window configured to receive a second component of an actuator.

Example 76. The stent of any example herein, particularly any of examples 73-74, wherein the actuator extends from the distal end of the stent towards the proximal end of the stent.

Example 77. The stent of any example herein, particularly any of examples 73-75, wherein the actuator comprises a lead screw and a nut and the nut is disposed within the window of the at least one vertical strut.

Example 78. The stent of any example herein, particularly any of examples 73-76, wherein the actuator comprises a locking mechanism.

Example 79. The stent of any example herein, particularly any of examples 73-77 wherein the actuator is configured to radially expand the stent from a radially compressed configuration to a radially expanded configuration.

Example 80. The stent of any example herein, particularly any of examples 74-79, wherein the actuator is configured to attach to an actuator of a prosthetic heart valve delivery apparatus.

Example 81. The stent of any example herein, particularly any of examples 65-80, wherein the at least one vertical strut of the plurality of vertical struts comprises at least one aperture.

Example 82. The stent of any example herein, particularly any of examples 65-81, wherein the at least one vertical strut of the plurality of vertical struts extends radially inwards from an outer of the stent.

Example 83. The stent of any example herein, particularly any of examples 65-82, wherein the frame comprises at least one commissure opening disposed between two adjacent angled struts or vertical struts, wherein the commissure opening extends radially inwards from the angled struts or vertical struts when the frame is in the radially expanded configuration.

Example 84. The stent of any example herein, particularly any of examples 65-83, further comprising a valvular structure attached to the stent and having a plurality of leaflets configured to allow blood to flow through the valvular structure from the distal end of the frame to the proximal end of the frame and preventing blood from flowing through the valvular structure from the proximal end of the frame to the distal end of the frame.

Example 85. The stent of any example herein, particularly any of examples 65-84, further comprising an outer skirt attached to the stent.

Example 86. The stent of any example herein, particularly any of examples 65-85, wherein the frame comprises two or more actuators and vertical struts configured to receive the actuators.

Example 87. The stent of any example herein, particularly any of examples 65-86, wherein the angled struts and the vertical struts are integrally formed as a unitary structure.

Example 88. The stent of any example herein, particularly any of examples 65-87, wherein the angled struts and the vertical struts are formed as a non-unitary structure.

Example 89. The frame of any of any example herein, particularly any of examples 65-89, wherein the plurality of angled struts and the plurality of vertical struts form a plurality of outer cells and a plurality of inner cells nested within the plurality of outer cells, wherein the plurality of inner cells is connected to the plurality of outer cells at one or more vertices of the inner cells.

Example 90. A medical assembly, comprising a frame having a first end portion, a second end portion, a longitudinal axis extending between the first end and the second end, a plurality of interconnected angled struts extending transverse to the longitudinal axis, and a plurality of vertical struts extending parallel to the longitudinal axis, an actuator, and a commissure opening, wherein the actuator is configured to radially expand the frame from a radially compressed configuration to a radially expanded configuration, wherein the plurality of interconnected angled struts and the plurality of vertical struts define a plurality of inner frame cells and a plurality of outer frame cells each having a distal apex and a proximal apex, wherein at least one vertical strut of the plurality of vertical struts comprises a first strut end, a second strut end, and at least one aperture, is configured to receive a first component of the actuator, is connected at the first strut end to the distal apex of an inner frame cell of the plurality of inner frame cells, extends toward the proximal apex of the inner frame cell is connected at the second end to a portion of the inner frame cell, and is heat set to deflect radially inwards from the inner frame cell while the frame is in the radially expanded configuration, wherein the commissure opening is disposed between adjacent outer frame cells and extends radially inwards from the plurality of interconnected angled struts and the vertical struts while the frame is in the radially compressed configuration.

Example 91. The medical assembly of any example herein, particularly example 90, wherein the at least one vertical strut of the plurality of vertical struts comprises an axially-extending channel to receive the first component of the actuator.

Example 92. The medical assembly of any example herein, particularly any of examples 90-91, wherein the actuator extends from the distal apex of an outer cell to a corresponding proximal apex of the outer cell.

Example 93. The medical assembly of any example herein, particularly any of examples 90-92, wherein the medical assembly comprises a valvular structure attached the frame and having a plurality of leaflets configured to allow blood to flow through the medical assembly from the first end portion of the frame to the second end portion of the frame and preventing blood from flowing through the medical assembly from the second end portion of the frame to the first end portion of the frame.

Example 94. The medical assembly of any example herein, particularly any of examples 90-93, wherein the medical assembly further comprises an outer skirt attached to the frame.

Example 95. The medical assembly of any example herein, particularly any of examples 90-94, wherein the actuator is configured to attach to an actuator of a prosthetic heart valve delivery apparatus.

Example 96. The medical assembly of any example herein, particularly any of examples 90-95, wherein the frame comprises two or more actuators and axially extending vertical struts configured to receive the actuators.

Example 97. A method for implanting a prosthetic heart valve, comprising plastically deforming a portion of a medical assembly having an annular frame while the annular frame is in a radially expanded configuration, heat setting the plastically deformed portion of the medical assembly, compressing the medical assembly from the radially expanded configuration to a radially compressed configuration, and releasing the medical assembly from the radially compressed configuration to a radially expanded configuration.

Example 98. The method of any example herein, particularly example 97, wherein the heat setting is accomplished by restraining the deformed portion of the medical assembly and exposing the portion of the medical assembly to an environment of over 450° C.

Example 99. The method of any example herein, particularly any of examples 97-98, wherein the medical assembly is advanced through the vasculature of a patient while in the radially compressed configuration.

Example 100. The method of any example herein, particularly any of examples 97-99, wherein the deformed portion of the medical assembly extends radially inwards from an outer circumference of the annular frame while the frame is in the radially expanded configuration.

Example 101. The method of any example herein, particularly any of examples 97-100, wherein the deformed portion of the medical assembly includes an axially extending vertical strut.

Example 102. The method of any example herein, particularly example 101, wherein the axially extending vertical strut is configured to receive an actuator.

Example 103. The method of any example herein, particularly example 102, wherein the actuator extends radially inwards from an outer diameter of the annular frame while the frame is in the radially expanded configuration.

Example 104. The method of any example herein, particularly any of examples 97-103, wherein the deformed portion of the medical assembly includes a commissure window that extends axially inwards from an outer diameter of the annular frame.

Example 105. A medical assembly, comprising a frame comprising a first end, a second end, a central longitudinal axis extending from the first end to the second end, a plurality of interconnected non-actuated struts, and at least one actuated strut extending parallel to the longitudinal axis and coupled to one or more non-actuated struts at a first strut end, and an actuator configured to radially expand and radially contract the frame, wherein the actuated strut comprises an axially-extending bore that receives a component of the actuator and a plurality of apertures disposed between the first strut end and a second strut end of the actuated strut.

Example 106. The medical assembly of any example herein, particularly example 105, wherein the actuator is a rotationally driven actuator.

Example 107. The medical assembly of any example herein, particularly example 106, wherein the actuator further comprises a screw thread.

Example 108. The medical assembly of any example herein, particularly example 107, wherein the interior of the axial bore comprises a screw thread configured to threadably engage with the screw thread of the actuator.

Example 109. The medical assembly of any example herein, particularly any of examples 105-108, wherein the frame comprises one or more additional actuators and corresponding axially extending vertical struts that each receive a component of the one or more additional actuators.

Example 110. The medical assembly of any example herein, particularly any of examples 105-109, wherein the medical assembly further comprises a valvular structure coupled to the frame and configured to permit blood to flow from the first end of the frame to the second end of the frame and to prevent blood from flowing from the second end of the frame to the first end of the frame.

Example 111. The medical assembly of any example herein, particularly any of examples 105-110, wherein the at least one actuated strut is heat set to extend inwards from the one or more interconnected struts when the frame is in the radially expanded configuration.

Example 112. The medical assembly of any example herein, particularly any of examples 105-110, further comprising one or more struts extending from the second strut end of the actuated strut to one or more interconnected struts of the plurality of interconnected non-actuated struts.

Example 113. The medical assembly of any example herein, particularly any of examples 105-110, wherein the second strut end of the actuated strut is connected to one or more interconnected struts of the plurality of interconnected non-actuated struts.

Example 114. The medical assembly of any example herein, particularly any of examples 105-110, wherein the frame further comprises a commissure opening disposed in one interconnected struts of the plurality of interconnected non-actuated struts.

Example 115. The medical assembly of any example herein, particularly example 114, wherein the strut having the commissure opening is head set to extend radially inwards from the adjacent interconnected struts of the frame.

Example 116. The medical assembly of any example herein, particularly any of examples 105-115, wherein the actuator is configured to releasably attach to a component of a valve delivery system.

Example 117. A prosthetic heart valve comprising a radially expandable frame comprising a first end portion, a second end portion, a longitudinal axis extending between the first end portion and the second end portion, a first post extending along the longitudinal axis, and a second post axially aligned with the first post, wherein the frame is radially movable between a radially compressed state and a radially expanded state; a valvular structure disposed within the frame and configured to regulate the flow of blood through the frame; and an actuation assembly operatively coupled to the frame, the actuation assembly comprising an actuator; wherein the first post is cantilevered and comprises a bore extending axially through the first post configured to receive the actuator, a first leg, and a second leg spaced laterally apart from the first leg to define an aperture extending radially through the first post.

Example 118. The prosthetic heart valve of claim 117, wherein the frame further comprises a plurality of struts arranged into a plurality of cells extending between the first end portion and the second end portion.

Example 119. The prosthetic heart valve of any example herein, particularly example 118, wherein the first post extends along the longitudinal axis and into a cell of the frame.

Example 120. The prosthetic heart valve any example herein, particularly any of examples 117-119, wherein the actuation assembly further comprises a nut and the first post further comprises a second aperture configured to receive the nut.

Example 121. The prosthetic heart valve any example herein, particularly any of examples 117-120, wherein the actuation assembly further comprises a stopper disposed between the first post and the second post.

Example 122. The prosthetic heart valve any example herein, particularly any of examples 117-121, wherein the actuator is rotatable relative to the first post and the second post to radially expand or radially compress the frame between the radially compressed state and the radially expanded state.

Example 123. The prosthetic heart valve any example herein, particularly any of examples 117-122, wherein the aperture has a diamond shape having a first axis extending from a first vertex of the aperture to a second vertex of the aperture and oriented along the longitudinal axis of the frame, and a second axis extending from a third vertex of the aperture to a fourth vertex of the aperture and oriented transverse to the longitudinal axis of the frame.

Example 124. The prosthetic heart valve any example herein, particularly any of examples 117-122, wherein the first leg and the second leg each comprise a first end portion, a second end portion, and a foldable joint between the first end portion and the second end portion.

Example 125. The prosthetic heart valve of any example herein, particularly example 124, wherein the foldable joint has a first angle when the frame is in the radially expanded state, and a second angle smaller than the first angle when the frame is in a radially compressed state.

Example 126. The prosthetic heart valve of any example herein, particularly example 125, wherein frame is radially compressible from the radially compressed state to a radially crimped state and the foldable joint has a third angle greater than the second angle when the frame is in the radially crimped state.

Example 127. The prosthetic heart valve any example herein, particularly any of examples 125-126, wherein the frame is radially expandable from the radially expanded state to a radially dilated state and the folded joint has a fourth angle smaller than the first angle when the frame is in the radially dilated state.

Example 128. The prosthetic heart valve any example herein, particularly any of examples 124-127, wherein the first leg and the second leg have a first thickness at the first end portion, a second thickness at the second end portion, and a third thickness at the joint, wherein the third thickness is less than the first thickness or the second thickness.

Example 129. The prosthetic heart valve any example herein, particularly any of examples 117-128, wherein the prosthetic heart valve comprises a plurality of cantilevered posts and a plurality of actuation assemblies operatively coupled to the plurality of cantilevered posts.

Example 130. The prosthetic heart valve any example herein, particularly any of examples 117-129, wherein the actuation assembly comprises a head portion configured to be releasably connected to a component of a delivery apparatus.

Example 131. A prosthetic heart valve comprising a radially expandable frame comprising a first end portion, a second end portion, a longitudinal axis extending between the first end portion and the second end portion, a first post extending along the longitudinal axis, and a second post axially spaced apart from first post, wherein the frame is radially expandable between a radially compressed state and a radially expanded state; a valvular structure disposed within the frame and configured to regulate the flow of blood through the frame; and an actuation assembly operatively coupled to the frame, comprising an actuator, a stopper, and a nut, wherein the first post is elastically deformable between a first state and a second state when the actuator imparts a force on the first post.

Example 132. The prosthetic heart valve of any example herein, particularly example 131, wherein the actuator is configured to impart a compressive force, such that the first post in the first state is axially longer than the first post in the second state.

Example 133. The prosthetic heart valve of any example herein, particularly example 132, wherein the actuator is further configured to impart a tensile force to elastically deform the first post from the second state to the first state.

Example 134. The prosthetic heart valve any example herein, particularly any of examples 131-133, wherein the actuator extends from the first post to the second post and through an axially oriented first bore in the first post and an axially oriented second bore in the second post.

Example 135. The prosthetic heart valve any example herein, particularly any of examples 131-133, further comprising an aperture extending radially through the first post, and the actuator extends axially from a first end portion of the aperture to a second end portion of the aperture.

Example 136. The prosthetic heart valve of any example herein, particularly example 135, wherein the aperture changes shape when the first post is elastically deformed from the first position to the second position.

Example 137. The prosthetic heart valve any example herein, particularly any of examples 131-135, wherein the first post comprises a nut window and the nut of the actuation assembly is disposed in the nut window.

Example 138. The prosthetic heart valve any example herein, particularly any of examples 131-134, wherein the stopper of the actuation assembly is positioned between the first post and the second post.

Example 139. The prosthetic heart valve any example herein, particularly any of examples 131-138, wherein when the prosthetic heart valve is configured to be deployable from a delivery sheath that compressively retains the prosthetic heart valve, and wherein the first post is in the first state while the prosthetic heart valve is retained by the delivery sheath.

Example 140. The prosthetic heart valve of any example herein, particularly example 139, wherein when the prosthetic heart valve is deployed from the delivery sheath, the first post is elastically deformed from the first state to the second state.

Example 141. The prosthetic heart valve of any example herein, particularly example 140, wherein the actuators may be rotated relative to the first post and the second post to radially expand the frame, and elastically deform the first post from the second state to the first state.

Example 142. The prosthetic heart valve of any example herein, particularly example 141, wherein the frame is further radially expandable by an inflatable balloon, and the first post elastically deforms from the first state to the second state when the frame is radially expanded by the inflatable balloon.

143. The prosthetic heart valve any example herein, particularly any of examples 131-142, wherein the prosthetic heart valve comprises a plurality of posts configured to elastically deform between a first state and a second state and a corresponding plurality of actuation assemblies operatively coupled to the plurality of posts.

Example 144. The prosthetic heart valve any example herein, particularly any of examples 131-142, wherein any deformation of the first post is elastic deformation.

Example 145. The prosthetic heart valve any example herein, particularly any of examples 131-144, wherein the actuation assembly comprises a head portion configured to be releasably connected to a component of a delivery apparatus.

Example 146. A medical assembly comprising a radially expandable frame comprising a first end portion, a second end portion, a longitudinal axis extending between the first end portion and the second end portion, a first post extending along the longitudinal axis, and a second post extending along the longitudinal axis wherein the frame is radially expandable between a radially compressed state and a radially expanded state; and an actuator extending from the first post to the second post; wherein the first post comprises a first window configured to change shape as the frame moves between the radially compressed state and the radially expanded state.

Example 147. The medical assembly of any example herein, particularly example 146, wherein the first window is defined by a first leg and a second leg spaced circumferentially apart from the second leg on the frame.

Example 148. The medical assembly of any example herein, particularly example 147, wherein the first leg and the second each comprise a first end portion and a second end portion separated by a foldable joint.

Example 149. The medical assembly of any example herein, particularly example 148, wherein the first leg and the second leg are joined at an end portion of each leg to form a diamond-shaped window with a first axis extending along the longitudinal axis of the frame and a second axis extending circumferentially between the first leg and the second leg.

Example 150. The medical assembly any example herein, particularly any of examples 147-149, wherein the foldable joint defines an angle and wherein the angle of the foldable joint is greater when the frame is in the radially expanded state than when the frame is in the radially compressed state.

Example 151. The medical assembly of any example herein, particularly example 150, wherein the frame is configured to be further radially compressed from the radially compressed state to a radially crimped state and wherein the angle of the foldable joint is greater in the radially crimped state than in the radially compressed state.

Example 152. The medical assembly of any example herein, particularly example 151, wherein the frame is configured to be further radially expanded from the radially expanded state to a radially dilated state and wherein the angle of the foldable joint is less in the radially dilated state than in the radially expanded state.

Example 153. The medical assembly any example herein, particularly any of examples 146-152, further comprising an internally threaded nut disposed around and engaged with an externally threaded portion of the actuator.

Example 154. The medical assembly of any example herein, particularly example 153, wherein the first post further comprises a second window sized to receive the internally threaded nut.

Example 155. The medical assembly any example herein, particularly any of examples 146-154, further comprising a stopper disposed around the actuator and positioned between the first post and the second post.

Example 156. The medical assembly any example herein, particularly any of examples 146-155, further comprising a valvular structure attached to the frame and configured to regulate the flow of blood in one direction.

Example 157. The medical assembly any example herein, particularly any of examples 146-156, wherein the frame further comprises a plurality of interconnected struts forming a plurality of cells, and wherein the first post is disposed within a cell of the plurality of cells.

Example 158. The medical assembly any example herein, particularly any of examples 146-157, wherein the medical assembly comprises a plurality of actuators operatively coupled to a corresponding plurality of struts having a first window configured to change shape as the frame moves between the radially expanded state and the radially compressed state.

Example 159. The medical assembly any example herein, particularly any of examples 146-158, wherein the actuator comprises a head portion configured to be releasably connected to an actuation member of a delivery apparatus.

160. A prosthetic heart valve comprising a radially expandable frame comprising a first end portion, a second end portion, a longitudinal axis extending between the first end portion and the second end portion, an axially compressible first post extending along the longitudinal axis and movable between an axially extended configuration and an axially compressed configuration, and a second post axially aligned with the first post; a valvular structure disposed within the frame and configured to regulate the flow of blood through the frame; and an actuation assembly operatively coupled to the frame, comprising an actuator extending between the first post and the second post, a stopper, and a nut, wherein the frame is radially movable from a radially compressed configuration to a radially expanded configuration and from a radially expanded configuration to a radially compressed configuration by rotating the actuators, wherein when the frame is in the radially expanded configuration, the first post is in the axially extended configuration, and wherein when the frame is in the radially compressed configuration, the first post is in the axially compressed configuration.

Example 161. The prosthetic heart valve of any example herein, particularly example 160, wherein the frame can be radially compressed from the radially compressed configuration to a radially crimped state, and wherein when the frame is in the radially crimped state, the first post is in the axially extended configuration.

Example 162. The prosthetic heart valve any example herein, particularly any of examples 160-161, wherein the frame can be radially expanded from the radially expanded configuration to a radially dilated configuration, and wherein when the frame is in the radially dilated configuration, the first post is in the axially compressed configuration.

Example 163. The prosthetic heart valve any example herein, particularly any of examples 160-162, wherein the first post further comprises a nut window that receives the nut of the actuation assembly.

Example 164. The prosthetic heart valve any example herein, particularly any of examples 160-163, wherein the stopper is disposed around the actuator and positioned between the first post and the second post.

Example 165. The prosthetic heart valve any example herein, particularly any of examples 160-164, wherein the first post comprises a collapsible aperture defined by a first leg and a second leg laterally spaced apart from each other and joined together at a first end portion and a second end portion of the first leg and the second leg.

Example 166. The prosthetic heart valve of any example herein, particularly example 165, wherein the aperture has a first axis extending axially along the first post and a second axis transverse to the first axis and extending circumferentially along the frame.

Example 167. The prosthetic heart valve of any example herein, particularly example 166, wherein when the first post is elastically deformed from the axially extended configuration to the axially compressed configuration, the aperture shortens along the first axis and lengthens along the second axis.

Example 168. The prosthetic heart valve of any one of any example herein, particularly any of examples 166-167, wherein when the first post is elastically deformed from the axially compressed configuration to the axially extended configuration, the aperture lengthens along the first axis and shortens along the second axis.

Example 169. The prosthetic heart valve of any one of any example herein, particularly any of examples 160-168, wherein the first post moves between the axially extended and the axially compressed configuration without plastically deforming.

Example 170. The prosthetic heart valve any example herein, particularly any of examples 160-169, wherein the actuation assembly comprises a head portion configured to be releasably connected to a component of a delivery apparatus.

Example 171. A frame for a medical assembly comprising a first end portion and a second end portion positioned along a longitudinal axis; and a first post extending from the first end portion along the longitudinal axis; wherein the frame is radially expandable between a radially compressed state and a radially expanded state, and wherein the first post comprises a first window configured to change shape as the frame moves between the radially compressed state and the radially expanded state.

Example 172. The frame of any example herein, particularly example 171, further comprising a second post extending along the longitudinal axis and axially aligned with the first post wherein an actuation assembly is operatively coupled to the first post and the second post.

Example 173. The frame of any example herein, particularly example 172, wherein the actuation assembly comprises an actuator extending between the first post and the second post, a stopper disposed around the actuator and positioned between the first post and the second post, and a nut.

Example 174. The frame of any example herein, particularly example 173, wherein the first post comprises a second window that receives the nut.

Example 175. The frame of any example herein, particularly example 174, wherein the second window is positioned between the first window and the second post.

Example 176. The frame any example herein, particularly any of examples 171-175, wherein a valvular structure is coupled to the frame and configured to allow the flow of blood through the frame in one axial direction and to prevent the flow of blood through the frame in the opposite axial direction.

Example 177. The frame any example herein, particularly any of examples 171-176, wherein when the frame moves from the radially compressed state to the radially expanded state, the window becomes longer along a direction parallel to the longitudinal axis.

Example 178. The frame any example herein, particularly any of examples 171-177, wherein when the frame moves from the radially expanded state to the radially compressed state, the window becomes longer along a direction perpendicular to the longitudinal axis.

Example 179. The frame any example herein, particularly any of examples 171-178, wherein the frame is further radially compressible from the radially compressed state to a radially crimped state and wherein when the frame moves from the radially compressed state to the radially crimped state, the window becomes longer along a direction parallel to the longitudinal axis of the frame.

Example 180. The frame any example herein, particularly any of examples 171-179, wherein the frame is further radially expandable from the radially expanded state to a radially dilated state and wherein when the frame moves from the radially expanded state to the radially dilated state, the window becomes longer along a direction perpendicular to the longitudinal axis of the frame.

Example 181. The frame any example herein, particularly any of examples 172-180, wherein the actuation assembly comprises a head portion configured to be releasably connected to a rotatable driver of a delivery apparatus.

Example 182. A prosthetic heart valve, comprising a radially expandable frame comprising a first end portion and a second end portion positioned along a longitudinal axis, a first post extending along the longitudinal axis, wherein the frame is movable between a radially compressed state and a radially expanded state; a valvular structure disposed within the frame and configured to regulate the flow of blood through the frame; and an actuator operatively coupled to the first post, wherein the first post is a mechanical fuse having a force threshold and configured to remain in a first state when the actuator imparts a force less than the force threshold to the first post, and to elastically deform from a first state to a second state when the actuator imparts a force greater than the force threshold on the first post.

Example 183. The prosthetic heart valve of any example herein, particularly example 182, wherein, when a compressive force imparted by the actuator above the force threshold is reduced below the force threshold, the mechanical fuse elastically deforms from the second state to the first state.

Example 184. The prosthetic heart valve any example herein, particularly any of examples 183-184, wherein the force threshold is greater than or equal to 25 N and less than or equal to 50 N.

Example 185. The prosthetic heart valve of any example herein, particularly example 184, wherein the force threshold is 40 N.

Example 186. The prosthetic heart valve any example herein, particularly any of examples 182-185, wherein the mechanical fuse has deformation threshold and wherein when an clastic deformation of the mechanical fuse is less than or equal to the deformation threshold, the mechanical fuse is in the first state and when an axial deflection of the mechanical fuse is greater than the elastic threshold, the mechanical fuse is in the second state.

Example 187. The prosthetic heart valve of any example herein, particularly example 186, wherein the deformation threshold is 0.05 mm.

Example 188. The prosthetic heart valve any example herein, particularly any of examples 182-187, wherein any deflection of the first post is elastic deformation.

Example 189. The prosthetic heart valve any example herein, particularly any of examples 182-188, further comprising a second post extending along the longitudinal axis and spaced apart from the first post along the longitudinal axis, wherein the actuator is operatively coupled to the second post.

Example 190. The prosthetic heart valve of any example herein, particularly example 189, wherein a stopper is disposed around the actuator and positioned between the first post and the second post.

Example 191. The prosthetic heart valve any example herein, particularly any of examples 182-190 wherein a nut is disposed around the actuator and wherein the first post comprises a window to receive the nut.

Example 192. The prosthetic heart valve any example herein, particularly any of examples 182-191 wherein the frame comprises a plurality of posts comprising a mechanical fuse and a corresponding number of actuators are operatively coupled to the plurality of posts.

Example 193. The prosthetic heart valve any example herein, particularly any of examples 182-192 wherein the actuator comprises a head portion configured to be releasably connected to a rotatable driver of a delivery apparatus.

Example 194. The prosthetic heart valve any example herein, particularly any of examples 182-193, further comprising a second post spaced axially apart from the first post, wherein the actuator is operatively coupled to the first post and the second post.

Example 195. A method for implanting a prosthetic heart valve, comprising, advancing the prosthetic heart valve, constrained by a component of a delivery apparatus, through the vasculature of the patient to a desired implantation site; deploying the prosthetic valve from a crimped state to a radially compressed state by removing the prosthetic valve from the component of the delivery apparatus; and radially expanding the prosthetic heart valve from the radially compressed state to a radially expanded state by rotating an actuator operatively coupled to a frame of the prosthetic heart valve relative to the frame of the prosthetic heart valve, wherein the prosthetic heart valve comprises a frame including an axially oriented first post configured to deflect between an axially extended state and an axially compressed state, wherein, when the prosthetic heart valve is deployed from the crimped state to the radially compressed state, the first post deflects from the axially extended state to the axially compressed state, and wherein, when the prosthetic heart valve is radially expanded from the radially compressed state to the radially expanded state, the first post deflects from the axially compressed state to the axially extended state.

Example 196. The method of any example herein, particularly example 195, wherein the prosthetic heart valve may further be radially dilated from the radially expanded state to a radially dilated state.

Example 197. The method of any example herein, particularly example 196, wherein when the prosthetic heart valve is radially dilated from the radially expanded state to the radially dilated state, the first post deflects from the axially extended state to the axially compressed state.

Example 198. The method any example herein, particularly any of examples 196-197, wherein the prosthetic heart valve is dilated from the radially expanded state to the radially dilated state by inflating an inflatable balloon positioned radially inwards of the frame of the prosthetic heart valve.

Example 199. The method any example herein, particularly any of examples 196-198, wherein the prosthetic heart valve is dilated from the radially expanded state to the radially dilated state as part of a valve in valve process.

Example 200. The method any example herein, particularly any of examples 195-199, wherein the component of the delivery apparatus is a delivery capsule.

Example 201. The method any example herein, particularly any of examples 195-199, wherein the component of the delivery apparatus is an adjustable loop.

Example 202. The method any example herein, particularly any of examples 195-201, wherein the first post comprises a collapsible aperture configured to change shape as the first post deflects from the axially extended state to the axially compressed state.

Example 203. The method of any example herein, particularly example 202, wherein the collapsible aperture is defined by a first leg and a second leg spaced axially apart.

Example 204. The method of any example herein, particularly example 203, wherein the first leg and the second each comprise a first end portion, a second end portion, and a foldable joint.

Example 205. The method of any example herein, particularly example 204, wherein the first end portion of the first leg is joined to the first end portion of the second leg, and the second end portion of the first leg is joined to the second end portion of the second leg to define a diamond-shaped aperture.

206. The method any example herein, particularly any of examples 204-205, wherein the first leg and the second leg each bend at the foldable joint when the first post deflects from the axially extended state to the axially compressed state or from the axially compressed state to the axially extended state.

Example 207. The method any example herein, particularly any of examples 195-206, wherein the deflection of the first leg between the axially extended and axially compressed state occurs solely by elastic deformation of the first leg.

Example 208. A method comprising sterilizing the prosthetic heart valve, the frame, the medical assembly, or the stent of any preceding example.

Example 209. An expandable frame, leaflets, or prosthetic heart valve of any preceding example, wherein the expandable frame, leaflets, or prosthetic heart valve is sterilized.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A frame for an implantable device, comprising:
a first end;
a second end;
a longitudinal axis extending from the first end to the second end;
a plurality of angled struts non-parallel to the longitudinal axis;
a plurality of vertically oriented struts extending parallel to the longitudinal axis and coupled to the angled struts at one or more vertical strut junctions; and
wherein one or more vertical struts of the plurality of vertically oriented struts includes a fixed end portion connected to a vertical strut junction, a free end portion, and a plurality of apertures disposed between the fixed end portion and the free end portion and configured to increase the flexibility of the one or more vertical struts.

2. The frame of claim 1, wherein at least one vertical strut of the plurality of vertically oriented struts comprises an axially extending channel extending from a first end of the vertical strut to a second end of the vertical strut configured to receive a first component of an actuator.

3. The frame of claim 2, wherein the vertical strut additionally comprises a window configured to receive a second component of the actuator and at least one aperture of the plurality of apertures is positioned between the fixed end portion of the vertical strut and the window.

4. The frame of claim 3, wherein at least one aperture of the plurality of apertures is positioned between the free end portion of the vertical strut and the window.

5. The frame of claim 2, wherein the actuator is configured to attach to an actuator of a prosthetic heart valve delivery apparatus.

6. The frame of claim 2, wherein the vertical strut extends radially inwards from a diameter of the frame when the frame is in a radially expanded configuration.

7. The frame of claim 2, wherein the frame comprises at least one stabilization strut extending from the free end portion of the at least one vertical strut to one of an angled strut or another vertical strut.

8. The frame of claim 1, wherein the frame comprises at least one commissure opening and the commissure opening extends radially inwards from a diameter of the frame when the frame is in a radially expanded configuration.

9. The frame of claim 1, further comprising a valvular structure attached the frame and having a plurality of leaflets configured to allow blood to flow through the valvular structure from the first end of the frame to the second end of the frame and to prevent blood from flowing through the valvular structure from the second end of the frame to the first end of the frame.

10. The frame of claim 1, further comprising an outer skirt attached to an exterior of the frame.

11. A prosthetic heart valve comprising:
a frame having a first end, a second end, a longitudinal axis extending from the first end to the second end, a plurality of angled struts extending transverse to the longitudinal axis, a plurality of vertical struts extending parallel to the longitudinal axis, and a plurality of junctions formed by an intersection of two or more angled struts, vertical struts, or a combination thereof;
a valvular structure having a plurality of leaflets configured to allow blood to flow through the prosthetic heart valve from the first end of the frame to the second end of the frame and to prevent blood from flowing through the prosthetic heart valve from the second end of the frame to the first end of the frame; and
wherein at least one vertical strut of the plurality of vertical struts has a fixed end and a free end, is attached at the fixed end to one junction of the plurality of junctions, and extends radially inwards from a diameter of the frame with a first radial displacement when the frame is in a radially expanded configuration.

12. The prosthetic heart valve of claim 11, wherein the at least one vertical strut of the plurality of vertical struts extends radially inwards from the diameter of the frame when the prosthetic heart valve is in a radially compressed configuration with a second radial displacement less than the first radial displacement.

13. The prosthetic heart valve of claim 11, wherein the at least one vertical strut does not extend radially inwards relative to the diameter of the frame when the prosthetic heart valve is in a radially compressed configuration.

14. The prosthetic heart valve of claim 11, wherein the at least one vertical strut of the plurality of vertical struts is shape set to radially extend inwards from the diameter of the frame when the prosthetic heart valve is in the radially expanded or partially radially expanded configuration.

15. The prosthetic heart valve of claim 11, wherein the frame comprises at least one stabilization strut extending from the free end of the at least one vertical strut to one of an angled strut or another vertical strut.

16. The prosthetic heart valve of claim 11, wherein the frame comprises at least one commissure opening wherein the commissure opening extends radially inwards the frame when the frame is in the radially expanded configuration.

17. The prosthetic heart valve of claim 11, wherein the frame comprises two or more vertical struts extending radially inwards from the plurality of vertical struts while the prosthetic heart valve is in the radially expanded configuration.

18. The frame of claim 11, wherein the plurality of angled struts and the plurality of vertical struts form a plurality of outer cells and a plurality of inner cells nested within the plurality of outer cells, wherein the plurality of inner cells are connected to the plurality of outer cells at one or more vertices of the inner cells.

19. A prosthetic heart valve comprising:
a radially expandable frame comprising:
a first end portion, a second end portion, a longitudinal axis extending between the first end portion and the second end portion, a first post extending along the longitudinal axis, and a second post axially aligned with the first post, wherein the frame is radially movable between a radially compressed state and a radially expanded state;
a valvular structure disposed within the frame and configured to regulate a flow of blood through the frame; and
an actuation assembly operatively coupled to the frame, the actuation assembly comprising an actuator,
wherein the first post is cantilevered and comprises a bore extending axially through the first post configured to receive the actuator, a first leg, and a second leg spaced laterally apart from the first leg to define an aperture extending radially through the first post.

20. The prosthetic heart valve of claim 19, wherein the frame further comprises a plurality of struts arranged into a plurality of cells extending between the first end portion and the second end portion.
